(19) United States
(12) Patent Application Publication
(10) Pub. No.: US 2025/0314593 A1
(43) Pub. Date: Oct. 9, 2025

(54) DEVICES, METHODS AND KITS FOR DETECTING EXPLOSIVES AND ILLICIT DRUG SUBSTANCES

(71) Applicant: Trace Eye-D, LLC, Bradenton, FL (US)

(72) Inventor: Bernard Gorski, Sarasota, FL (US)

(21) Appl. No.: 18/836,632

(22) PCT Filed: Feb. 7, 2023
(86) PCT No.: PCT/US2023/012551

§ 371 Date: Aug. 7, 2024

Related U.S. Application Data
(60) Provisional application No. 63/307,389, filed on Feb. 7, 2022.

(51) Int. Cl. G01N 21/78; G01N 1/02; G01N 1/10

(52) U.S. Cl. CPC G01N 21/78; G01N 1/10; G01N 2001/022; G01N 2001/028

(57) ABSTRACT

A wipe for detecting the presence of an explosive or illicit drug substance is composed of an absorbent or adsorbent substrate and a chemical detection solution impregnated within the substrate. The chemical detection solution includes a combination of reagents operable, when contacted with a particular explosive substance or illicit drug undergo a chemical reaction or a series of chemical reactions to produce a compound having a visible color. The wipes can be sealed within heat sealed pouches that contain small amounts of reagents necessary to test a surface, article or sample. Individual, disposable wipe delivery systems present the opportunity to cross over into many field detection applications for explosives, drug detection, and other applications in the area of forensics or environmental detection, where transportability, ease of use, and safety are desired. Kits and methods for detecting the presence of a substance include or utilize at least one chemical detection solution and at least one wipe.

### BACKGROUND OF THE DISCLOSURE

The present invention relates to devices, methods, and kits for rapidly detecting explosives and illicit drugs, and more particularly to devices, methods and kits configured to chemically detect the presence or absence of a bulk amount of explosives or illicit drugs or a trace residue of explosives or illicit drugs on a surface.

The statements in this section merely provide background information related to the present disclosure and should not be construed as constituting prior art.

To ensure the safety of individuals working in various industries, such as the travel, law enforcement and parcel industries, the ability to rapidly identify and detect indicators of explosive materials or illicit drugs is extremely important. Examples of indicators of explosive materials include, for example and without limitation, compounds present in pre-detonated explosive materials such as reagents, intermediates and other chemicals used to make explosive materials (collectively referred to as “precursor compounds”); impurities commonly found in precursor compounds or explosive materials; explosive materials themselves; and post-detonated explosive residues such as chemical products and byproducts of an explosion or detonation (such indicators collectively referred to herein as “explosive substances” or, singularly, an “explosive substance”). Drug discovery and interdiction of illicit substances has become increasing dangerous, with the advent of potent and deadly forms of fentanyl now present. While various detection techniques have been described over time, these techniques are largely insufficient for a variety of reasons. Colorimetric detection is often the first line discovery used for first responders or law enforcement officers. However, such personnel may need to wear physical protection, such as gloves and face shields making the manipulation of sampling equipment and reactant tubes are hard to handle. Moreover, there is a need for a small compact kit that does not present potential chemical hazards in the case of breakage, that can be easily carried and stored. Others have commercialized kits that produce colorimetric reactions using the collection and manipulation of samples collected and the introduction of reagents by breaking glass ampoules, using bottled sprays, or introduction liquid droplets, all contained within larger liquid storage containers.

There remains a need for a convenient and accurate on-site detection system that can rapidly detect the presence or absence of dangerous substances, either explosives or illicit drugs or other chemicals in an operational environment without the need for additional detection equipment and/or materials and volumes of potential hazardous liquid reagents. The present disclosure addresses this need.

### SUMMARY OF THE DISCLOSURE

Devices, kits and products described herein have a unique operational capability. Many embodiments provide for performance of an assay as a one step process following removal of a towellete or other wipe from a package, and do not require the use of ampoules, sprays, or droplets. In many embodiments, the sample is collected wet on a wipe, which is very easy to use and provides for good sample collection efficiency. In many embodiments, reagents are contained within two or more separate compartments of a single sealed package and the reagents are activated by simple mixing within the package shortly before the package is opened and the wipe is removed for use. In many embodiments, the wipes have high sensitivities and provide instantaneous or nearly instantaneous response. Moreover, as the wipe substrates contain relatively small amounts of reagents, they can be readily shipped, and do not require special handling protocols. They could, for example, be sent to any country as an item in a diplomatic pouch for US embassy protection in foreign countries. In many embodiments, the wipes do not represent a Resource Conservation and Recovery Act (RCRA) hazardous material, and therefore do not need special waste management after use or at the end of their shelf life.

In accordance with one aspect of the present disclosure, a wipe for detecting the presence of an explosive or illicit drug substance is provided that comprises a fibrous substrate (also referred to herein as an absorbent or adsorbent substrate and as a “wipe”) and a chemical detection solution impregnated into the substrate. The chemical detection solution includes a combination of reagents operable, when contacted with a particular substance to undergo a chemical reaction or a series of chemical reactions to produce a compound having a characteristic visible color. In certain preferred embodiments, a wipe as described herein is sealed within a single-use pouch and the chemical detection solution comprises relatively small amounts of reagents operable to test an article, a surface or a solid or liquid sample. Such a product, referred to herein as a “packaged wipe,” can be individually packaged in an easy-to-use pouch, much like a common alcohol wipe. Use of such a wipe avoids the need to use glass vials or droppers to add reagents to a wipe, as the reactants are impregnated in the sealed wipe or in an adjacent small chamber or compartment in binary packaging systems, which is activated into the substrate for use when the pouch is opened. Such a product can be readily shipped without hesitation, as only small quantities of reactant are contained in each pouch and this does not present a shipping hazard. This unique delivery system presents the opportunity to cross over into many field detection applications for explosives and drug detection where transportability, ease of use, and safety are important requirements. Such a delivery system also presents opportunities to cross into other field detection applications including, for example and without limitation, colorimetric kits for military, environmental, engineering, or medical applications.

In some embodiments described herein, packaged wipes are provided that are made such that certain reagents are kept separated within a package and then mixed with one another shortly before the wipe is to be used. Certain combinations of reagents that are useful to detect a given target substance, such as an explosive substance, an illicit drug substance or the like, might interact or react with one another over time after being mixed together, thereby causing degradation of one or more reagent over time, or causing a reaction that diminishes the effectiveness of the chemical detection solution over time. Thus, if the combination of reagents is packaged in a fully pre-mixed form, such interactions or reactions can detract from the reliability of the product over time and diminish the shelf-life of the product. Moreover, one or more of the reagents may cause degradation of the substrate wipe itself if it is impregnated within the wipe within a package, which also would detract from the reliability of the product over time and diminish the shelf life of the product. In one form of the disclosure, therefore, there is provided a multi-chamber or multi-compartment packaging system for packaging a chemical detection wipe in a manner whereby one or more reagent in a chemical detection solution is kept separated from one or more other reagent in the chemical detection solution until shortly before the detection wipe is to be used. Such a detection wipe packaging system provides enhanced stability and shelf-life to a detection wipe product and provides other benefits. In one embodiment, a packaged detection wipe comprises a package defining a first sealed compartment and a second sealed compartment, a fibrous substrate positioned in the first compartment, a first precursor component of a chemical detection solution positioned in the first compartment with, and some embodiments impregnated within, the fibrous substrate, and a second precursor component of a chemical detection solution positioned in the second compartment, wherein the first and second compartments are separated from one another by a frangible seal. In this embodiment, mixture of the first precursor component and the second precursor component provides a combination of reagents that comprises the complete chemical detection solution.

In one form of the disclosure, for example an embodiment in which the packaged wipe is a chlorate/perchlorate-detection wipe, the two compartments of a package can contain two separate chemical detection solutions that are themselves operable to detect different target substances. For example, in a chlorate/perchlorate embodiment, one chemical detection solution that includes dimethyl sulfoxide, an alcohol, an acid and diphenylamine to detect a chlorate is contained in the first compartment of the package with the wipe and another chemical detection solution that comprises an aqueous solution of methylene blue to detect a perchlorate is positioned in the second compartment of the package. In one embodiment, the alcohol comprises isopropyl alcohol. In another embodiment, the acid comprises sulfuric acid. In still another embodiment, the sulfuric acid comprises 30% sulfuric acid.

In another form, in which the packaged wipe is for detection of hydrogen peroxide or an organic peroxide such as TATP or MEKP (collectively referred to as peroxides), the chemical detection solution includes a redox color indicating agent (also referred to herein as an internal redox indicator) that is configured to exhibit a color change when the solution contacts a particular explosive substance. In one embodiment, the internal redox indicator is a pH independent redox indicator. In another embodiment, the internal redox indicator is a pH dependent redox indicator. In alternative embodiments, the internal redox indicator is substituted with a colored inorganic oxidant or a colored inorganic reductant. In one specific embodiment, the internal redox indicator comprises diphenylamine.

In yet another form, in which the packaged wipe is for the detection of nitrate esters, nitrosamines and other nitrogen-based explosive substances (referred to herein as a “nitro-detection wipe”), the combination of reagents includes Griess reagents. As used herein, the term “Griess reagents” is used to refer to reagents operable to perform a Griess test, which is an analytical chemistry test for detecting the presence of a nitrite ion.

In one form of the disclosure, in which the packaged wipe is for cocaine detection, the chemical detection solution includes cobalt thiocyanate, glycerin, and an acid. In other embodiments, the chemical detection solution further includes one or both of hydrochloric acid and chloroform. Suitable descriptions of such chemical detection solution embodiments are described in Tsumura, Y.; Mitome, T.; Kimoto, S. “False positives and false negatives with a cocaine-specific field test and modification of test protocol to reduce false decision.” Forensic Science International. 2005, 155 (2-3), 158-164, which is incorporated herein by reference in its entirety.

In another form, in which the packaged wipe is a methamphetamine/ecstasy detection wipe, the chemical detection solution includes a sodium nitroprusside used as a test for the presence of alkaloids common in illicit substances. Suitable descriptions of such chemical detection solution embodiments are described in “Color Test Reagents/Kits for Preliminary Identification of Drugs of Abuse NIJ Standard 0604.01” Prepared for: National Institute of Justice Office of Science and Technology Washington, DC 20531 July 2000 NCJ 18325. The test, called Simon's test, is performed by adding a solution of sodium nitroprusside and acetaldehyde in deionized water along the addition of an aqueous sodium carbonate solution. The test turns blue for secondary amines encountered that are used as illicit simulants including 3,4-methylenedioxymethamphetamine (MDMA, the main component in Ecstasy) and phenethylamines such as methamphetamine.

In yet another form, in which the packaged wipe is for the detection of fentanyl, the chemical detection solution includes eosin Y reagent dissolved in phosphate buffered in deionized water with a small amount of yellow food coloring. As used herein, the term “eosin Y reagent” is used to refer to which is an analytical chemistry test for detecting the presence of fentanyl.

The fibrous substrate can take a wide variety of forms, provided that the substrate is operable to absorb, adsorb or otherwise become impregnated with the chemical detection solution. The fibrous substrate in preferred embodiments has a light color so that a color change of the chemical detection solution is readily visible and most preferably is white or nearly white in color. According to certain embodiments herein, the fibrous substrate comprises a plurality of fibers that are selected from the group consisting of cellulose, polyamides, polyesters, polyethylenes, polypropylenes, polyacrylics, cellulose acetate, polylactic acid, silk, wool, glass, polyaramids, and combinations thereof. In accordance with still other embodiments, the fibrous substrate is a nonwoven material that has multiple layers formed from fibers selected from the group consisting of polyolefin, polypropylene, polyethylene, ethylene copolymers and propylene copolymers. According to yet still other embodiments, the fibrous substrate is an absorbent nonwoven material that is formed from a blend of spunbonded fibers selected from the group consisting of polypropylene, polyester, and wood pulp.

In one chlorate/perchlorate-detection wipe embodiment, a first part of the chemical detection solution that is contained within the first compartment with the fibrous substrate comprises dimethyl sulfoxide, isopropyl alcohol, sulfuric acid, and diphenylamine. In another embodiment, the first part of chemical detection solution has an acidic pH. In yet another embodiment, the first part of the chemical detection solution has a pH of from about 1.0 to about 6.0. In another embodiment, the first part of the chemical detection solution has a pH of from about 2.5 to about 5.0. In one embodiment, the sulfuric acid has a concentration of from about 1.5M to about 11.0M. In another embodiment, the sulfuric acid has a concentration of from about 5.4M to about 5.8M. In another embodiment, the sulfuric acid has a concentration of about 2.55M. In another embodiment, the sulfuric acid has a concentration of about 5.6M. In still another embodiment, the chemical detection solution comprises from about 6 to about 9% dimethyl sulfoxide (by weight), from about 4 to about 8% isopropyl alcohol (by weight), from about 0.1 to about 2% diphenylamine (by weight) and from about 80% to about 90.0% by weight of sulfuric acid (30% solution). A 100 ml volume of the 30% solution of sulfuric acid can be prepared, for example, by diluting about 26 ml of 98% sulfuric acid in about 60 ml of water. In still yet another embodiment, the first part of the chemical detection solution comprises about 7.4% dimethyl sulfoxide (by weight), about 6.0% isopropyl alcohol (by weight), about 0.5% diphenylamine (by weight) and about 86.0% by weight of sulfuric acid. It is not intended, however, that the present disclosure be limited to this specific alcohol, acid or redox color indicating agent, a variety of alternate alcohols, acidic fluids and redox color indicating agents being well known and readily available. The second part of the chemical detection formula is comprised of methylene blue.

In one nitro-detection wipe embodiment, the chemical detection solution comprises a carrier fluid and a combination of reagents operable to undergo a chemical reaction or a series of chemical reactions to produce a compound having a visible color when the solution contacts the explosive substance, the combination of reagents includes Griess reagents. In another embodiment, the Griess reagents include sulfanilamide and an ethylenediamine dihydrochloride compound.

In one peroxide-detection wipe embodiment, the chemical detection solution comprises a carrier fluid, a redox color indicating agent having a first reduction potential and at least one member of a redox pair having a second reduction potential. In another embodiment, the first reduction potential and the second reduction potential sufficiently correlate to one another to enable the redox color indicating agent to produce a color change when the solution contacts a particular explosive substance (referred to herein as “correlated reduction potentials”). In one embodiment, the carrier fluid has an acidic pH. In another embodiment, the carrier fluid has a pH of from 0 to about 3.5. In yet another embodiment, the carrier fluid has a pH of from about 0.5 to about 1.5. In still another embodiment, the carrier fluid includes an acidic aqueous fluid and at least one organic solvent. In still yet another embodiment, the acidic aqueous fluid includes hydrochloric acid and sulfuric acid. It is not intended, however, that the present disclosure be limited to these specific acids, a variety of alternate acidic fluids being well known and readily available. In still another embodiment, the at least one organic solvent includes dimethyl sulfoxide, isopropyl alcohol and dipropylene glycol dimethyl ether. It is not intended, however, that the present disclosure be limited to these specific organic solvents, a variety of alternate organic solvents being well known and readily available. In still another embodiment, the redox color indicating agent includes diphenylamine and the at least one member of a redox pair includes ferric ions, which can be provided in the solution, for example, in the form of ferric chloride.

In another aspect, the present disclosure provides a method of fabricating an explosive or illicit drug detection wipe that comprises (i) providing a chemical detection solution that includes a carrier fluid and a combination of reagents operable to undergo a chemical reaction or a series of chemical reactions to produce a compound having a visible color when the solution contacts the explosive or illicit drug residue, (ii) providing a fibrous substrate, and (iii) impregnating the fibrous substrate with the chemical detection solution to provide an impregnated substrate. In one embodiment, the method further includes placing the impregnated substrate in a sealed package. When the method is used to fabricate a chlorate/perchlorate-detection wipe embodiment, a first chemical detection solution is one that is operable to produce a compound having a visible color when the solution contacts a chlorate and a second chemical detection solution is one that is operable to product a visible color when the second solution contacts a perchlorate substance, multiple embodiments of which are disclosed herein. When the method is used to fabricate a hydrogen peroxide-detection wipe embodiment, the chemical detection solution is one that is operable to produce a compound having a visible color when the solution contacts hydrogen peroxide or an organic peroxide such as TATP or MEKP, multiple embodiments of which are disclosed herein. When the method is used to fabricate a nitro-detection wipe embodiment, the chemical detection solution is one that is operable to produce a compound having a visible color when the solution contacts nitrate esters, nitroamines and other nitrogen-based explosive substances (referred to collectively herein as “nitro explosive substances”), multiple embodiments of which are disclosed herein. When the method is used to fabricate a fentanyl-detection wipe embodiment, the chemical detection solution is one that is operable to produce a compound having a visible color when the solution contacts a fentanyl residue, multiple embodiments of which are disclosed herein. When the method is used to fabricate a methamphetamine-detection wipe embodiment, the chemical detection solution is one that is operable to produce a compound having a visible color when the solution contacts a methamphetamine residue, multiple embodiments of which are disclosed herein. When the method is used to fabricate a cocaine-detection wipe embodiment, the chemical detection solution is one that is operable to produce a compound having a visible color when the solution contacts cocaine residue, multiple embodiments of which are disclosed herein.

In another aspect of the disclosure, there are provided methods of testing a surface or a solid or liquid sample for an explosive or illicit drug substance. In one embodiment, the method includes (i) providing a first detection wipe fabricated from a fibrous substrate, (ii) introducing a first chemical detection solution onto the first wipe, the first chemical detection solution including at least one reagent operable to undergo a chemical reaction or a series of chemical reactions to produce a compound having a visible color when the solution contacts a first explosive or illicit drug substance, (iii) wiping the surface or contacting the solid or liquid sample with the first detection wipe, (iv) observing whether the first detection wipe exhibits a change in color, and (v) determining whether the first explosive or illicit drug substance is present on the surface or in the solid or liquid sample based on whether the first detection wipe exhibits a color change. When the method is used to test a surface or a solid or liquid sample for a chlorate or perchlorate compound, the first chemical detection solution is one that is operable to produce compounds having a visible color when the solution contacts a chlorate or perchlorate compound, multiple embodiments of which are disclosed herein. When the method is used to test a surface for a peroxide, the first chemical detection solution is one that is operable to produce a compound having a visible color when the solution contacts hydrogen peroxide or an organic peroxide such as Triacetone triperoxiide (TATP) or methyl ethyl ketone peroxide (MEKP), multiple embodiments of which are disclosed herein. When the method is used to test a surface for nitrate esters, nitroamines and other nitrogen-based explosive substances, the first chemical detection solution is one that is operable to produce a compound having a visible color when the solution contacts nitrate esters, nitroamines and other nitrogen-based explosive substances, multiple embodiments of which are disclosed herein. When the method is used to test a surface for fentanyl, the first chemical detection solution is one that is operable to produce a compound having a visible color when the solution contacts fentanyl, multiple embodiments of which are disclosed herein. When the method is used to test a surface for methamphetamine, the first chemical detection solution is one that is operable to produce a compound having a visible color when the solution contacts methamphetamine, multiple embodiments of which are disclosed herein. When the method is used to test a surface for cocaine, the first chemical detection solution is one that is operable to produce a compound having a visible color when the solution contacts cocaine, multiple embodiments of which are disclosed herein.

In another embodiment, the method further includes (vi) providing a second detection wipe fabricated from a fibrous substrate, (vii) introducing a second chemical detection solution onto the second wipe, the second chemical detection solution at least one reagent operable to undergo a chemical reaction or a series of chemical reactions to produce a compound having a visible color when the solution contacts a second explosive or illicit drug substance, (viii) wiping the surface or contacting the solid or liquid sample with the second detection wipe, (ix) observing whether the second detection wipe exhibits a change in color, and (x) determining whether the second explosive or illicit drug substance is present on the surface or in the solid or liquid sample based on whether the second detection wipe exhibits a color change. In one embodiment, at least one of the first explosive substance and the second exposive substance is selected from the group consisting of a chlorate or perchlorate, hydrogen peroxide or an organic peroxide such as TATP or MEKP, a nitro explosive substance, cocaine, methamphetamine and a fentanyl residue. In another embodiment, at least one of the first and second chemical detection solutions is selected from the group consisting of a chemical detection solution comprising dimethyl sulfoxide, an alcohol, an acid and diphenylamine; an aqueous solution of methylene blue; a chemical detection solution comprising Griess reagents; a chemical detection solution that includes dimethyl sulfoxide, isopropyl alcohol, diphenylamine, sulfuric acid, dipropylene glycol dimethyl ether, ferric chloride and hydrochloric acid; a chemical detection solution comprising cobalt thiocyanate, an acid, glycerine, and optionally one or both of hydrochloric acid or chloroform; a chemical detection solution comprising acetaldehyde, sodium nitroprusside and sodium carbonage; and a chemical detection solution comprising an aqueous solution of eosin Y.

In another embodiment, the method further includes (xi) providing a third detection wipe fabricated from a fibrous substrate, (xii) introducing a third chemical detection solution onto the third wipe, the third chemical detection solution including at least one reagent operable to undergo a chemical reaction or a series of chemical reactions to produce a compound having a visible color when the solution contacts a third explosive or illicit drug substance, (xiii) wiping the surface or contacting the solid or liquid sample with the third detection wipe, (xiv) observing whether the third detection wipe exhibits a change in color, and (xv) determining whether the third explosive or illicit drug substance is present on the surface or in the solid or liquid sample based on whether the third detection wipe exhibits a color change. In one embodiment, at least one of the first explosive or drug substance (referred to herein as “target substance”), the second target substance and the third target substance is selected from the group consisting of a chlorate or perchlorate, hydrogen peroxide or an organic peroxide such as TATP or MEKP, a nitro explosive substance, cocaine, methamphetamine or fentanyl. In another embodiment, at least two of the first target substance, the second target substance and the third target substance is selected from the group consisting of a chlorate or perchlorate, hydrogen peroxide or an organic peroxide such as TATP or MEKP, a nitro explosive substance, fentanyl, cocaine or methamphetamine. In yet another embodiment, each of the first target substance, the second target substance and the third target substance is selected from the group consisting of a chlorate or perchlorate, hydrogen peroxide or an organic peroxide such as TATP or MEKP, a nitro explosive substance, fentanyl, methamphetamine and cocaine. In still another embodiment, each of the first, second and third chemical detection solutions is selected from the group consisting of a chemical detection solutions comprising dimethyl sulfoxide, an alcohol, an acid and diphenylamine; an aqueous solution of methylene blue; a chemical detection solution comprising Griess reagents; a chemical detection solution that includes dimethyl sulfoxide, isopropyl alcohol, diphenylamine, sulfuric acid, dipropylene glycol dimethyl ether, ferric chloride and hydrochloric acid; a chemical detection solutions that includes cobalt thiocyanate, glycerin and optionally one or more of an acid and chloroform; a chemical detection solution that includes an alcohol, acetaldehyde and sodium nitro prusside; and a chemical detection solution comprising a solution of eosin Y.

In yet another aspect of the disclosure, a method of testing a surface or a solid or liquid sample for an explosive or illicit drug substance includes (i) providing a detection wipe fabricated from a fibrous substrate, (ii) contacting the detection wipe with the surface or the solid or liquid sample, (iii) introducing onto the wipe a chemical detection solution as disclosed herein that includes a combination of reagents operable, when contacted with the explosive substance to undergo a chemical reaction or a series of chemical reactions to produce a compound having a visible color, (iv) observing whether the detection wipe exhibits a change in color, and (v) determining whether the explosive substance is present on the surface or in the solid or liquid sample based on whether the detection wipe exhibits a color change. When the method is used to test a surface or a solid or liquid sample for a chlorate or perchlorate chemical, the chemical detection solution is one that is operable to produce a compound having a visible color when the solution contacts a chlorate or perchlorate chemical, multiple embodiments of which are disclosed herein. When the method is used to test a surface for a peroxide, the chemical detection solution is one that is operable to produce a compound having a visible color when the solution contacts hydrogen peroxide or an organic peroxide such as TATP or MEKP, multiple embodiments of which are disclosed herein. When the method is used to test a surface for nitrate esters, nitroamines and other nitrogen-based explosive substances, the chemical detection solution is one that is operable to produce a compound having a visible color when the solution contacts nitrate esters, nitroamines and other nitrogen-based explosive substances, multiple embodiments of which are disclosed herein. When the method is used to test a surface or substance for fentanyl the chemical detection solution is one that is operable to produce a compound having a visible color change when the solution contacts fentanyl, multiple embodiments of which are disclosed herein. When the method is used to test a surface or substance for methamphetamine, the chemical detection solution is one that is operable to produce a compound having a visible color when the solution contacts methamphetamine, multiple embodiments of which are disclosed herein. When the method is used to test a surface or substance for cocaine the chemical detection solution is one that is operable to produce a compound having a visible color when the solution contacts cocaine, multiple embodiments of which are disclosed herein. In additional embodiments of the method, actions (i)-(iv) are repeated one or more times, but using in a successive repetition a chemical detection solution that is operable to test a surface for a different type of explosive or drug substances. In this way, the method is used to test a surface for multiple types of explosive or drug substances using different types of chemical detection solutions as disclosed herein. A variety of additional methods and modes for determining whether an explosive or drug substance is present on a surface also are disclosed herein, as will be appreciated upon consideration of the entire disclosure herein.

In another aspect of the disclosure, there is provided a packaged wipe that is operable to detect the presence of an explosive substance or an illicit drug substance, the packaged wipe comprising (i) a flexible package including a first seal surrounding a first compartment and a second compartment and including a second seal between the first compartment and the second compartment, (ii) a fibrous substrate positioned in the first compartment, (iii) a first precursor component of a chemical detection solution positioned in the first compartment and impregnated within the fibrous substrate, and (iv) a second precursor component of the chemical detection solution positioned in the second compartment. In some embodiments, the second seal is a frangible seal configured to be breached upon application to the first compartment or the second compartment a compressive force of a magnitude sufficient to breach the frangible seal but insufficient to breach the first seal, thereby permitting the first precursor component and the second precursor component to mix. Mixture of the first precursor component and the second precursor component produces a chemical detection solution operable, when contacted with the explosive substance or the illicit drug substance, to undergo a chemical reaction or a series of chemical reactions to produce a compound having a characteristic visible color

In another aspect, the present disclosure provides kits to test for a plurality of diverse target substances on a surface or in a solid or liquid sample, which target substances may comprise one or more explosive substances, one or more illicit drugs or a combination thereof, or even other substances. In one embodiment, the kit includes at least two wipes for detecting different target substances. In one embodiment, the kit includes at least two wipes for detecting different explosive substances. In another embodiment, the kit includes at least two wipes for detecting different illicit drug substances. In another embodiment, the kit includes at least one wipe for detecting an explosive substance and at least one wipe for detecting an illicit drug substance. In another embodiment, each of the two wipes is operable to produce a compound having a visible color when the solution contacts at least one explosive substance selected from a chlorate, a perchlorate, hydrogen peroxide, an organic peroxide such as TATP or MEKP and a nitro substance, or at least one illicit drug selected from fentanyl, methamphetamine and cocaine.

In one form, a kit comprises at least two wipes selected from the group consisting of: (i) a first wipe contained within a compartment of a first sealed package, the first wipe impregnated with a first chemical detection solution or a portion thereof, wherein the first chemical detection solution is operable to produce a compound having a visible color when the solution contacts a first explosive or drug illicit substance; (ii) a second wipe contained within a compartment of a second sealed package, the second wipe impregnated with a second chemical detection solution or a portion thereof; wherein the second chemical detection solution is operable to produce a compound having a visible color when the second chemical detection solution contacts a second explosive or illicit drug substance different than the first explosive or illicit drug substance; and (iii) a third wipe contained within a compartment of a third sealed package, the third wipe impregnated with a third chemical detection solution or a portion thereof; wherein the third chemical detection solution is operable to produce a compound having a visible color when the third chemical detection solution contacts a third explosive or illicit drug substance different than the first and second explosive or illicit drug substances. In embodiments in which one or more of the first, second or third wipes is impregnated with only a portion of a corresponding chemical detection solution, it is understood that the corresponding sealed package also includes a second compartment containing a second portion of the corresponding chemical detection solution, with the compartments separated by a frangible seal that can be breached to mix the portions together, thereby activating the respective chemical detection solution.

In one embodiment of the kit, at least one of the first and second chemical detection solutions is operable to produce a compound having a visible color when the solution contacts an explosive substance selected from chlorate or perchlorate. In another embodiment, the chemical detection solution comprises dimethyl sulfoxide, an alcohol, an acid, and diphenylamine. In yet another embodiment, the chemical detection solution has a pH of from about 1.0 to about 6.0. In another embodiment, the chemical detection solution has a pH of from about 2.5 to about 5.0. In still yet another embodiment, the chemical detection solution comprises from about 6 to about 9% dimethyl sulfoxide (by weight), from about 4 to about 8% isopropyl alcohol (by weight), from about 0.1 to about 2% diphenylamine (by weight) and from about 80% to about 90.0% by weight of sulfuric acid. In one embodiment, the sulfuric acid of the chemical detection solution has a concentration of from about 1.5M to about 11.0M. In another embodiment, the sulfuric acid has a concentration of from about 5.4M to about 5.8M. In another embodiment, the sulfuric acid has a concentration of about 2.55M. In another embodiment, the sulfuric acid has a concentration of about 5.6M. In another embodiment the kit further includes a second chemical detection solution that comprises an aqueous solution of methylene blue for the detetion of perchlorate.

In another embodiment of the kit, at least one of the first and second wipes is operable to produce a compound having a visible color when the solution contacts an explose substance selected from hydrogen peroxide or an organic peroxide such as TATP or MEKP. In yet another embodiment, the at least one of the first and second wipes is sealed in a package defining at least two compartment, a first compartment containing a fibrous substrate and a first precursor component of the chemical detection solution, the first precursor component comprising dipropylene glycol dimethyl ether, ferric chloride and hydrochloric acid. A second compartment of the package contains a second precursor component of the chemical detection solution, the second precursor component comprising dimethyl sulfoxide, isopropyl alcohol, diphenylamine and sulfuric acid.

In yet another embodiment of the kit, the at least one of the first and second chemical detection solutions (or optionally a third chemical detection solution of a third wipe in a kit embodiment) is operable to produce a compound having a visible color when the solution contacts an explosive substance comprising a nitro explosive substance. In still another embodiment, a first precursor component of the third chemical detection solution comprises ethylene diamine dihydrochloride, dimethyl sulfoxide, tetrabutylammonium hydroxide and isopropyl alcohol; and wherein the second precursor component of the third chemical detection solution comprises an aqueous solution of sulfanilamide, butanol, zinc powder and phosphoric acid. In still yet other embodiments, the first precursor component of the third chemical detection solution comprises dimethyl sulfoxide, tetrabutylammonium hydroxide, ethylenediamine dihydrochloride and isopropyl alcohol; and the second precursor component of the third chemical detection solution comprises sulfanilamide and phosphoric acid. In further embodiments, either the first precursor component or the second precursor component further comprises butanol and zinc powder.

In one embodiment of the kit, at least one of the first and second chemical detection solutions (or optionally a third chemical detection solution of a third wipe in a kit embodiment) is operable to produce a compound having a visible color when the solution contacts an illicit drug comprising fentanyl. In one embodiment, the chemical detection solution of the fentanyl detection wipe comprises eosin Y in an aqueous solution. In another embodiment, the chemical detection solution comprises from about 0.5 to about 1.5 percent solution of eosin Y in a buffered aqueous solution with a pH ranging from about 6 to about 9, with a small amount of yellow food coloring added to enhance the color change. In one embodiment, the solution is buffered using a phosphate buffer.

In another embodiment of the kit, at least one of the first and second chemical detection solutions (or optionally a third chemical detection solution of a third wipe in a kit embodiment) is operable to produce a compound having a visible color when the solution contacts an illicit drug comprising methamphetamine. In one embodiment, the chemical detection solution of the methamphetamine detection wipe comprises Simon's reagent. In one embodiment, the chemical detection solution is separated into a first precursor component comprising an aqueous solution of sodium nitroprusside and acetaldehyde and a second precursor component comprising an aqueous solution of sodium carbonate.

In yet another embodiment of the kit, at least one of the first and second chemical detection solutions (or optionally a third chemical detection solution of a third wipe in a kit embodiment) is operable to produce a compound having a visible color when the solution contacts an illicit drug comprising cocaine. In one embodiment, the chemical detection solution of the cocaine detection wipe comprises cobalt thiocyanate, acetic acid, glycerine, and optionally one or more of hydrochloric acid or chloroform. In one embodiment, the chemical detection solution is separated into a first precursor component comprising cobalt thiocyanate, and glycerine and a second precursor component comprising one or more of an acid, such as acetic acid and/or hydrochloric acid, and/or chloroform.

In another form of the disclosure, a kit to test for a plurality of diverse target substances on a surface is provided that includes (i) a wipe contained within a first compartment of a sealed package, the wipe impregnated with a first chemical detection solution comprising dimethyl sulfoxide, an alcohol, an acid and diphenylamine; wherein the first chemical detection solution is operable to produce a compound having a visible color when the solution contacts a chlorate; the sealed package further defining a second compartment that contains second chemical detection solution comprising an aqueous solution of methylene blue, which is operable to produce a compound having a visible color when the solution contacts perchlorate (ii) a container; and (iii) a mixture contained within the container, the mixture comprising dipropylene glycol dimethyl ether, ferric chloride and hydrochloric acid; wherein a mixture of the first chemical detection solution with the mixture produces a third chemical detection solution operable to produce a compound having a visible color when the solution contacts hydrogen peroxide or an organic peroxide such as TATP or MEKP.

Still other features, characteristics, objects, and benefits of the disclosure will become apparent from the following description.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is a schematic plan view of a packaged detection wipe product in accordance with certain embodiments;

FIG. 2 is a schematic plan view of a packaged detection wipe product in accordance with certain embodiments;

FIG. 3 is a cross-sectional view of a heat sealable multilayer material used to make a package in accordance with certain embodiments;

FIG. 4 is a schematic plan view of a kit in accordance with certain embodiments; and

FIG. 5 is a schematic plan view of a kit in accordance with certain embodiments.

### DETAILED DESCRIPTION

The embodiments of the present application described below are not intended to be exhaustive or to limit the teachings of the present application to the precise forms disclosed in this detailed description. Rather, the embodiments are chosen and described so that others skilled in the art may appreciate and understand the principles and practices of the present application.

Unless defined otherwise, all technical and scientific terms used herein have the same meaning as commonly understood by one of ordinary skill in the art to which this application belongs. Moreover, it should be understood that when certain values and ranges are recited herein in connection with various embodiments of the present teachings, all values and ranges which fall between such listed values and ranges are intended to be encompassed by the present teaching unless explicitly stated otherwise. Finally, although specific methods and materials are described herein with respect to certain aspects of the present disclosure, it should be understood and appreciated that other methods and materials similar or equivalent to those described herein can be used in the practice or testing of the present application without straying from the invention's intended scope.

As will be explained herein, the present disclosure provides devices that include a chemical detection solution impregnated in a substrate, the chemical detection solution operable to selectively react or interact with and visibly identify trace and bulk amounts of explosive or illicit drug substances. Trace amounts of explosives or drugs are defined as invisible chemical residues resulting from handling, packaging or carrying a substance, while bulk amounts are defined as a visible amounts. In accordance with certain aspects of this disclosure, the substrate is in the form of a wipe that is packaged as a single unit for individual use. In other embodiments, multiple wipes are packaged together and can be packaged, for example, in a container operable to dispense wipes individually for use, if desired, a wide variety of which are commercially available. In still other embodiments, kits are provided that can be used to test a surface for a variety of different explosive or drug substances.

The substrate can be fabricated from any suitable absorbent or adsorbent material, such as a textile material comprising a plurality of yarns provided in a knit or woven construction or a plurality of fibers that are provided in a non-woven construction. The fibrous substrate can take a wide variety of forms, provided that the substrate is operable to absorb, adsorb or otherwise become impregnated with the chemical detection solution. For example, the fibrous substrate can be of the type commonly used in Kimwipes™ products (Kimberly-Clark), Clorox® wipe products (The Clorox Company), baby wipe products, paper towel products and the like. The fibrous substrate in preferred embodiments has a light color so that a color change of the chemical detection solution is readily visible, and most preferably the fibrous substrate is white or nearly white in color. Suitable fibers include, but are not limited to, cellulose (e.g., cotton and rayon), polyamides, polyesters, polyethylenes, polypropylenes, polyacrylics, cellulose acetate, polylactic acid, silk, wool, glass, polyaramids, and combinations thereof. In specific illustrative embodiments, the substrate comprises an absorbent nonwoven material, such as a bonded and carded material, a spunbonded material, or a meltblown material including meltblown microfibers. In accordance with certain aspects herein, the nonwoven material may also have multiple layers such as, for example, multiple spunbonded layers and/or multiple meltblown layers. Moreover, the nonwoven material may be made of polymers such as, for example, polyolefins, which are intended to include polypropylene, polyethylene, ethylene copolymers and propylene copolymers. According to certain embodiments, the nonwoven material may be an elastic nonwoven material, while in accordance with other embodiments a non-elastic nonwoven material or an extensible nonwoven material may be used.

In one representative embodiment, the substrate is composed of a blend of spunbonded polypropylene, polyester and wood pulp. In one embodiment, the substrate comprises an absorbent nonwoven material including from about 15% to about 35% spunbonded polypropylene, from about 10% to about 30% polyester and from about 40% to about 60% wood pulp, all by weight. In another embodiment, the substrate comprises an absorbent nonwoven material including from about 22% to about 32% spunbonded polypropylene, from about 17% to about 27% polyester and from about 46% to about 56% wood pulp, all by weight. In yet another embodiment, the absorbent nonwoven material may comprise about 26.7% spunbonded polypropylene, about 22.2% polyester and about 51.1% wood pulp, all by weight. As those of skill in the art will understand and appreciate, the production of fibrous layers by means of spun bonding is based on the direct spinning of polymeric granulates into continuous filaments and subsequently manufacturing the fibrous layer. Spun bond fabrics are produced by depositing extruded, spun fibers onto a moving belt in a uniform random manner followed by thermal bonding the fibers. The fibers are separated during the web-laying process by air jets, and fiber bonds are generated by applying heated rolls or hot needles to partially melt the polymer and fuse the fibers together. Since molecular orientation increases the melting point, fibers that are not highly drawn can be used as thermal binding fibers, and polyethylene or random ethylene/-propylene copolymers can be used as low melting bonding sites.

As used herein, the term “polymer” generally includes, but is not limited to, homopolymers, copolymers, such as, for example, block, graft, random and alternating copolymers, terpolymers, etc., and blends and modifications thereof. Furthermore, unless otherwise specifically limited, the term “polymer” includes all possible geometrical configurations of the material. These configurations include, but are not limited to, isotactic, syndiotactic and random symmetries.

To detect the presence or absence of an explosive or illicit drug substance on the surface of an object or in a solid or liquid sample, the device is contacted with the surface or the solid or liquid sample and, if the target explosive or illicit drug substance is present, the chemical detection solution impregnated in the substrate reacts or interacts with the explosive or illicit drug substance and produces a representative color change that can be visibly observed by the user without the need for special equipment (e.g., a spectrometer) or the aid of an intervening processing step (e.g., conversion of color change into an electronic signal that is processed by an interpreting device).

To cause the color change to occur, the chemical detection solution includes a combination of reagents operable, when contacted with a particular explosive or illicit drug substance to undergo a chemical reaction or a series of chemical reactions to produce a compound having a characteristic visible color. Multiple combinations of reagents are disclosed herein that can be employed to undergo a chemical reaction or a series of chemical reactions to produce a compound having a visible color when contacted with a particular explosive or illicit drug substance. In one form of the disclosure, in which the wipe is a wipe for the detection of nitrate esters, nitroamines and other nitrogen-based explosive substances (referred to herein as a “nitro-detection wipe”), a suitable combination of reagents includes Griess reagents. As used herein, the term “Griess reagents” refers to reagents operable to perform a Griess test, which is an analytical chemistry test for detecting the presence of a nitrite ion, as described further below. In the presence of nitrite ions, the Griess reagents and the nitrite ion undergo a series of reactions that ultimately produce a compound having visible color, such as an azo dye. While Griess reagents can be used to detect a variety of nitrate ester compounds and nitroamine compounds, a person of ordinary skill in the art will recognize that the chemical detection solution according to this embodiment must also operate to either isolate nitrite ions from the nitrate ester compounds and/or nitroamine compounds, so that the Griess reagents and the nitrite ions can interact to produce the compound having visible color, or react with nitro functional groups of the nitrate ester compounds, nitroamine compounds or other nitro compounds, for example, to convert sulfanilamide to a diazonium salt. The formulations disclosed herein have been found to have excellent detection properties for a wide variety of nitrate esters, nitroamines and other nitro compounds that are known to be explosive compounds and/or to be byproducts of explosions of nitrogen-based explosive materials.

Thus, in one useful application of the disclosed invention, the explosive substances being detected are nitrogen-based explosives. As used herein, the term “nitrogen-based explosives” refers to explosive materials, compounds used to make explosive materials and products of detonation of explosive materials that include nitrate ester moieties, nitroamine moieties and/or other nitro groups (referred to herein as “nitrate ester compounds,” “nitroamine compounds” and “other nitro compounds,” respectively, or “nitro compounds,” collectively). The formulations disclosed herein that are useful for making nitro-detection wipes are operable to interact with nitrite ions released from the nitrate ester compound, nitroamine compound or other nitro compound upon contact with a chemical detection solution as contemplated by the present disclosure or, in some instances, to interact with a nitro functional group of certain compounds. Examples of nitrate ester compounds include, without limitation, nitroglycerin, nitrocellulose (including guncotton), erythritol tetranitrate (ETN) and pentaerythritol tetranitrate (PETN). Examples of nitroamine compounds include, without limitation, HMX and RDX. An example of other nitro compound includes, without limitation, trinitrotoluene (TNT). Examples of products that include combinations of these nitrogen-based explosive include, without limitation, Semtex, which is a combination of RDX and PETN, and Composition B (Comp B), which is a combination of RDX and TNT.

In one nitro detection-embodiment, the chemical detection solution includes a carrier fluid and Griess reagents. In another embodiment, the chemical detection solution includes sulfanilamide and an ethylenediamine hydrochloride compound. In one embodiment, the ethylenediamine dihydrochloride compound comprises ethylenediamine dihydrochloride. In another embodiment, the ethylenediamine dihydrochloride compound comprises N-(1-naphthyl)ethylenediamine dihydrochloride. It is not intended, however, that the present disclosure be limited to these specific compounds, a variety of alternate ethylenediamine dihydrochloride compounds being known and readily available.

In another embodiment the carrier fluid has an acidic pH. In yet other embodiments, the carrier fluid has a pH of from about 1.0 to about 6.0. In another embodiment, the carrier fluid has a pH of from about 2.5 to about 5.0 or from about 3.0 to about 4.5 or from about 3.5 to about 4.0 or about 3.75. In still another embodiment, the carrier fluid includes an acidic aqueous fluid and at least one organic solvent. In still yet another embodiment, the acidic aqueous fluid includes phosphoric acid. In another embodiment, the at least one organic solvent includes dimethyl sulfoxide. It is not intended, however, that the present disclosure be limited to this specific organic solvent, a variety of alternate organic solvents being well known and readily available. For example, and without limitation, another organic solvent contemplated by this disclosure includes acetone. In another embodiment, the carrier fluid includes an alcohol. In yet another embodiment, the alcohol comprises methanol. It is not intended, however, that the present disclosure be limited to this specific alcohol, a variety of alternate alcohols being well known and readily available. For example, and without limitation, another alcohol contemplated by this disclosure includes ethanol. In another embodiment, the carrier fluid includes a base. In one embodiment, the base comprises tetrabutylammonium hydroxide (TBAH). It is not intended, however, that the present disclosure be limited to this specific base, a variety of alternate bases, including bases having similar strengths to TBAH, being well known and readily available.

In one embodiment, the chemical detection solution includes dimethyl sulfoxide, methanol, TBAH, sulfanilamide, ethylenediamine dihydrochloride and phosphoric acid. In another embodiment, the chemical detection solution includes from about 65% to about 85% dimethyl sulfoxide, from about 5% to about 13% methanol, from about 1% to about 5% TBAH, from about 3% to about 9% sulfanilamide, from about 0.1% to about 0.3% ethylenediamine dihydrochloride and from about 5% to about 10% phosphoric acid, all by weight.

In another form of the disclosure, in which the wipe is a wipe for the detection of hydrogen peroxide or an organic peroxide such as TATP or MEKP (referred to herein as a “peroxide-detection wipe”), the chemical detection solution includes a redox color indicator that is operable to change color when the solution comes into contact with hydrogen peroxide or an organic peroxide such as TATP or MEKP (if present). As those of skill in the art will understand and appreciate, redox color indicating agents are intended to refer to those materials that can undergo a redox reaction, and thereby change color, when exposed to appropriate conditions. In the embodiments directed to peroxide-based wipes, such conditions arise when reagents in the solution come into contact with hydrogen peroxide or an organic peroxide such as TATP or MEKP. As a result of the redox reaction, the redox color indicating agents exhibit a color change. Examples of redox color indicators that are contemplated in accordance with various embodiments of the present disclosure include, but are not limited to, neutral red, amino black, safranine T or O, indigo, indigo carmine, methylene blue, thionine, thymol indophenol, gallocyanine, nile blue, variamine blue, diphenylamine, 2,6-dichlorophenolindophenol, diphenylamine-4-sulfonic acid, barium salt, tris(2,2-dipyridyl)iron(II)sulfate, N-phenylanthranilic acid, ferroin, nitroferroin, 5,6-dimethylferroin, 4-amino-4′-methyldiphenylamine, diphenylbenzindine-disulfonic acid, o-dianisidine, 3,3′-dimethylnaphthidine, 3,3′-dimethylnaphthidine disulfonic acid, bis(5-bromo-1,10-phenanthroline)ruthenium(II)dinitrate, tris(5-nitro-1,10-phenanthroline)iron(II)sulfate, Iron(II)-2,2′,2″-tripyridine sulfate, tris(4,7-biphenyl-1,10-phenanthroline)iron(II)disulfate, o,m′-diphenylaminedicarboxylic acid setopaline, p-nitrodiphenylamine, tris(1,10-phenanthroline)-iron(II) sulfate, setoglaucine O, xylene cyanole FF, erioglaucine A, eriogreen, tris(2,2′-bipyridine)-iron(II)hydrochloride, 2-carboxydiphenylamine[N-phenyl-anthranillic acid], benzidine dihydrochloride, o-toluidine, bis(1,10-phenanthroline)-osmium(II) diphenylamine-4-sulfonate Na salt), 3,3′-dimethoxybenzidine dihydrochloride[o-dianisidine], ferrocyphen, 4′-ethoxy-2,4-diaminoazobenzene, N,N-diphenylbenzidine, diphenylamine, N,N-dimethyl-p-phenylenediamine, variamine blue B hydrochloride, N-phenyl-1,2,4-benzenetriamine, bindschedler's green, 2,6-dichloroindophenol (Na salt), 2,6-dibromophenolindophenol, brilliant cresyl blue [3-amino-9-dimethyl-amino-10-methylphenoxyazine chloride], Iron(II)-tetrapyridine chloride, starch (soluble potato, 13 present), gallocyanine (25° C.), nile blue A [aminonaphthodiethylamino-phenoxazine sulfate], Indigo-5,5′,7,7′-tetrasulfonic acid (Na salt), Indigo-5,5′,7-trisulfonic acid (Na salt), Indigo-5,5′-disulfonic acid (Na salt), phenosatranine, indigo-5-monosulfonic acid (Na salt), bis(dimethylglyoximato)-iron(II)chloride, Induline scarlet, and the like.

As utilized herein, “color change” or “change in color” refers to a change in light absorption, reflection, or fluorescence which can be observed visually or with the help of a simple instrument such as a small ultraviolet light. The term “light” refers to electromagnetic radiation in ultraviolet, visible, near infrared and infrared wavelength ranges.

In one embodiment, the chemical detection solution includes a carrier fluid and a redox color indicating agent. In another embodiment, the redox color indicating agent comprises diphenylamine and the chemical detection solution includes ferric ions. In yet another embodiment, the redox color indicating agent comprises diphenylamine, the chemical detection solution includes ferric ions and the chemical detection solution includes an acidic carrier fluid.

In one hydrogen peroxide-detection wipe embodiment, the chemical detection solution comprises a carrier fluid, a redox color indicating agent having a first reduction potential and at least one member of a redox pair having a second reduction potential. In another embodiment, the first reduction potential and the second reduction potential sufficiently correlate to one another to enable the redox color indicating agent to produce a color change when the solution contacts a particular explosive substance (referred to herein as “correlated reduction potentials”). In one embodiment, the carrier fluid has an acidic pH. In another embodiment, the carrier fluid has a pH of from 0 to about 3.5. In yet another embodiment, the carrier fluid has a pH of from about 0.5 to about 1.5. In still another embodiment, the carrier fluid includes an acidic aqueous fluid and at least one organic solvent. In still yet another embodiment, the acidic aqueous fluid includes hydrochloric acid and sulfuric acid. It is not intended, however, that the present disclosure be limited to these specific acids, a variety of alternate acidic fluids being well known and readily available. In still another embodiment, the at least one organic solvent includes dimethyl sulfoxide, isopropyl alcohol and dipropylene glycol dimethyl ether. It is not intended, however, that the present disclosure be limited to these specific organic solvents, a variety of alternate organic solvents being well known and readily available. In still another embodiment, the redox color indicating agent includes diphenylamine and the at least one member of a redox pair includes ferric ions, which can be provided in the solution, for example, in the form of ferric chloride.

In one embodiment, the chemical detection solution is formed from a first solution that includes dipropylene glycol dimethyl ether, ferric chloride, and hydrochloric acid and a second solution that includes dimethyl sulfoxide, isopropyl alcohol, diphenylamine, and sulfuric acid. In one embodiment, the first solution and the second solution are combined in a volumetric ratio of from about 2:1 to about 1:2 to provide a final solution, which is the chemical detection solution. In yet another embodiment, the chemical detection solution is formed from a first solution that includes from about 95% to about 99% dipropylene glycol dimethyl ether, from about 0.1% to about 1.5% ferric chloride and from about 0.01% to about 0.5% hydrochloric acid (30% solution), all by weight, and a second solution that includes from about 6% to about 9% dimethyl sulfoxide, from about 4% to about 8% isopropyl alcohol, from about 0.1% to about 2% diphenylamine and from about 80% to about 90% sulfuric acid (50% solution), all by weight. In still another embodiment, the chemical detection solution is formed from a first solution that includes about 98.4% dipropylene glycol dimethyl ether, about 0.8% ferric chloride and about 0.073% hydrochloric acid (30% solution), all by weight, and a second solution that includes about 7.4% dimethyl sulfoxide, about 6.0% isopropyl alcohol, about 0.5% diphenylamine and about 86.0% sulfuric acid (50% solution), all by weight.

While the hydrochloric acid is described in the above embodiments as being composed of a 30% solution, it is understood that the concentration of the hydrochloric acid can vary from this concentration without departing from the disclosure, and that other embodiments employ hydrochloric acid having different concentrations. For example, in one embodiment, the hydrochloric acid has a concentration of from about 5% to about 80%. In another embodiment, the hydrochloric acid concentration is selected such that the HCl in the first solution has a molarity of from about 4 to about 8, and in still other embodiments the HCl in the first solution has a molarity of from about 5 to about 7 or from about 5.5 to about 6.5. Similarly, while the sulfuric acid is described in the above embodiments as being composed of a 50% solution, it is understood that the concentration of the sulfuric acid can vary from this concentration without departing from the disclosure, and that other embodiments employ sulfuric acid having different concentrations. For example, in one embodiment, the sulfuric acid has a concentration of from about 10% to about 70%. In another embodiment, the sulfuric acid has a concentration of about 30%. In yet another embodiment, the sulfuric acid concentration is selected such that the sulfuric acid in the second solution has a molarity of from about 3 to about 8, and in still other embodiments the sulfuric acid in the second solution has a molarity of from about 4 to about 7 or from about 5 to about 6.

A person of ordinary skill in the art will appreciate from the present disclosure that the proportions of the components or ingredients of the chemical detection solutions disclosed herein can be varied beyond the example proportions set forth herein and that such variations may impact the speed with which the color-emitting compound is produced or the redox color indicating agent exhibits a color change when it comes into contact with the explosive substance, the amount or concentration of explosive substance required to produce a visible color change within the detection device or other property of the device.

In another form of the disclosure, in which the wipe is a wipe for the detection of a chlorate (referred to herein as a “chlorate-detection wipe”), the chemical detection solution includes dimethyl sulfoxide, an alcohol, an acid and diphenylamine and is operable to change color when the solution comes into contact with a chlorate (if present). In certain preferred embodiments, a chlorate detection wipe may be packaged in a package that defines a second compartment containing a chemical detection solution operable to detect a perchlorate. In one embodiment, the second chemical detection solution contained in a second compartment is an aqueous solution of methylene blue and is operable to change color when the solution comes in contact with a perchlorate (if present). In one embodiment, the acid in the chemical detection solution for chlorate is an acid having a pKa of from about −2 to about −5. In another embodiment, the chemical detection solution for chlorate includes dimethyl sulfoxide, isopropyl alcohol, sulfuric acid, and diphenylamine. In yet another embodiment, the chemical detection solution for chlorate comprises from about 6 to about 9% dimethyl sulfoxide (by weight), from about 4 to about 8% isopropyl alcohol (by weight), from about 0.1 to about 2% diphenylamine (by weight) and from about 80% to about 90.0% by weight of sulfuric acid. In still another embodiment, the chemical detection solution for chlorate comprises about 7.4% dimethyl sulfoxide (by weight), about 6.0% isopropyl alcohol (by weight), about 0.5% diphenylamine (by weight) and about 86.0% by weight of sulfuric acid. In one chlorate/perchlorate-detection wipe embodiment, the chemical detection solution in the first compartment for chlorate has a pH of from about 1.0 to about 6.0. In another embodiment, the chemical detection solution in the first compartment for chlorate has a pH of from about 2.5 to about 5.0. In another embodiment, the chemical detection solution for chlorate comprises sulfuric acid at a concentration of from about 1.5M to about 11.0M. In another embodiment, the sulfuric acid has a concentration of from about 5.4M to about 5.8M. In yet another embodiment, the chemical detection solution for chlorate in the first compartment comprises sulfuric acid at a concentration of about 2.55M. In another embodiment, the sulfuric acid has a concentration of about 5.6M. The sulfuric acid in one embodiment is sulfuric acid having a concentration of about 50% and in another embodiment is sulfuric acid having a concentration of about 30%. Alternatively, sulfuric acid of other concentrations can be used, such as, for example, sulfuric acid at a concentration of from about 10% to about 70%. Acid concentrations can be lowered by diluting stock solutions of the respective acids with deionized water to provide an acid having a desired concentration. A sulfuric acid having a concentration of about 30% has been shown to provide a good balance of function and stability in the devices, methods and kits described herein; however, this disclosure is not limited to this concentration. In the above embodiments, the chemical detection solution in the second compartment for perchlorate is an aqueous solution of methylene blue.

In another form of the disclosure, in which the wipe is a wipe for the detection of a fentanyl (referred to herein as a “fentanyl-detection wipe”), the chemical detection solution eosin Y, and is operable to change color when the solution comes into contact with a fentanyl (if present). In one embodiment, the chemical detection solution comprises from about 0.5 to about 1.5 percent solution of eosin Y in a buffered aqueous solution with a pH of from about 6 to about 9, with a small amount of yellow food coloring added to enhance the color change. In one embodiment, the solution is buffered using a phosphate buffer. In other embodiments, other buffer systems are used.

In another form of the disclosure in which the wipe is a wipe for the detection of methamphetamine (referred to herein as a “methamphetamine-detection wipe”), the chemical detection solution is formed from a first solution that includes an aqueous solution of sodium nitroprusside and acetaldehyde and a second solution that include an aqueous solution of sodium carbonate. The first solution and the second solution are combined in a volumetric ratio of 1:1 to provide a final solution, which is the chemical detection solution.

In another form of the disclosure in which the wipe is a wipe for the detection of cocaine (referred to herein as a “cocaine-detection wipe”), the chemical detection is formed from a single solution of “Scott's reagent.” In one embodiment, the Scott's reagent includes cobalt thiocyanate, 30% acetic acid, glycerin, and optionally hydrochloric acid and/or chloroform.

In accordance with certain exemplary illustrations herein, the substrate can come in several forms, including, but not limited to, tissues, pads, cloths, sheets, wipes, and towelettes. Moreover, the chemical detection solution is composed of a formulation that is designed to molecularly react or interact with a specific targeted detection substance or group of substances. For instance, in accordance with certain aspects herein, the substrate can be impregnated with a specific chemical formulation that is designed to interact with specific explosive or illicit drug material, such as a residue of nitrogen based explosive materials, a residue of peroxide based explosive materials or chlorate/perchlorate. Examples of explosive materials that are intended to be included by the various methods and techniques of the present teachings include, but are not necessarily limited to, TNT, Octol, 1,3,5 triazido 2,4,6 trinitrobenzene (TATNB), 4,4′ Dinitro-3,3′diazenofuroxan, trinitroaniline, tetryl, picric acid, Dunnite, methyl picrate, ethyl picrate, picryl chloride, trinitrocresol, lead styphnate, triaminotrinitrobenzene, 1,1 diamino-2-2-dinitrothene, Hydrogen peroxide, Triaceteone triperoxide (TATP), HMTD, MEKP, Methyl Nitrate, Potassium nitrate, sodium nitrate, urea nitrate ammonium nitrate, ANFO, EGDN Mannitol hexanitrate, PETN, ETN, Ethylenedinitramine, nitroguanidine, RDX (C4, C2), HMX, tetranitramine, Hexanitrohexaazoisowurtzitane, sorguyl, Potassium perchlorate, sodium perchlorate, potassium chlorate, sodium perchlorate, Semtex, and guncotton as well as most gun powders. Examples of illicit drug materials that are intended to be included by various methods and techniques of the present teaching include, but are not necessarily limited to cocaine, cocaine hydrochloride, methamphetamine, Ecstasy, and fentanyl.

The present disclosure contemplates a wide variety of methods to form a detection wipe as disclosed herein. One method includes (i) providing a chemical detection solution in accordance with the present disclosure, (ii) providing a fibrous substrate, and (iii) impregnating the fibrous substrate with the chemical detection solution. In another embodiment, the method further includes sealing the substrate in a package. In alternate embodiments, the fibrous substrate can be impregnated with the chemical detection solution before the fibrous substrate is positioned in the package or after the fibrous substrate is positioned in the package and prior to the sealing of the substrate in the package. In one embodiment, the method further includes flushing the package with an inert gas, such as, for example, carbon dioxide or nitrogen gas after the substrate is positioned in the package and before the package is sealed to reduce the amount of oxygen gas within the package or to remove oxygen gas from the package prior to sealing.

To make a packaged detection wipe in accordance with certain embodiments, the substrate can be fed from storage rolls onto a coating machine, where the explosive or illicit drug detection solution, or portion thereof, is applied. In alternative embodiments, which are provided only as examples, the solution can be added by running the substrate through a trough of the solution or the solution can be added to the substrate by spraying the formula from a series of nozzles. In still other embodiments, individual towelettes or wipes may be packaged in sealed foil pouches or packages by a process in which sheets of laminated foil are fed into a machine that folds them into a small pouch and heat seals three sides to form an open envelope. Simultaneously, another conveyor line feeds the substrates into the pouch, while a liquid feed mechanism injects the chemical detection solution into the envelope containing the towelettes or wipes. Once the solution is added, another heat sealer then closes the remaining side of the pouch to seal the impregnated substrate within the package in a ready-to-use form.

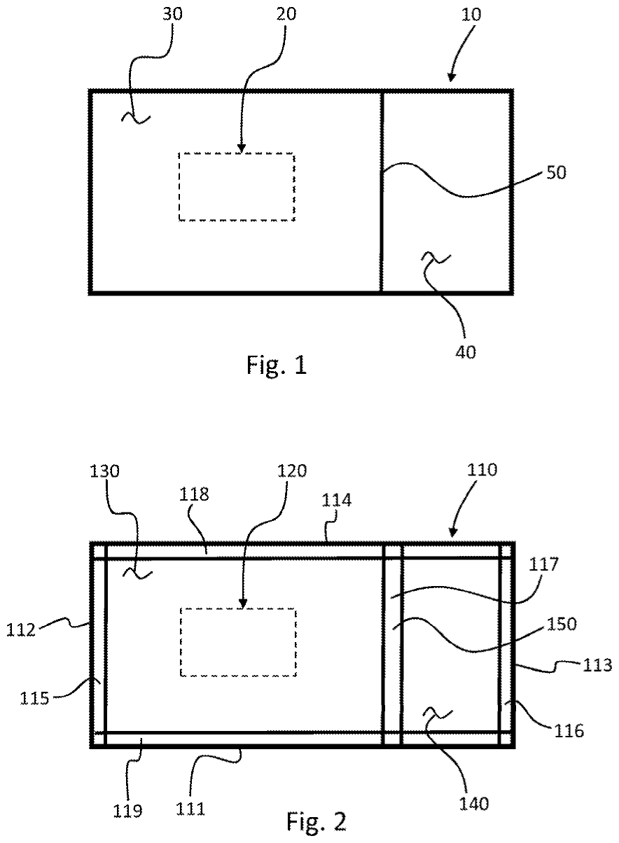

Additional variations of packaged detection wipes also are envisioned to provide enhanced stability and shelf-life to a detection wipe product and to provide other benefits. In one example, a packaged detection wipe is provided in which two or more precursor components of the chemical detection solution are packaged in a manner in which the precursor components are isolated from one another during storage and mixed together shortly before the wipe is used. In one embodiment, depicted schematically in FIG. 1, this is accomplished by positioning fibrous substrate 20 impregnated with a first precursor component of a chemical detection solution in first compartment 30 of package 10 that is composed of a flexible material, such as, for example, a multilayer foil laminate material, and positioning a second precursor component of the chemical detection solution in second compartment 40 of package 10 wherein first and second compartments 30, 40 are separated by frangible seal 50. In one embodiment, frangible seal 50 is configured such that a user can breach the frangible seal by applying a compressive force on package 10 that is of a magnitude sufficient to breach frangible seal 50 but less than a magnitude that would breach the package itself. In this way, a user can breach frangible seal 50 by squeezing package 10, thus permitting the first and second precursor components of the chemical detection solution to be combined. As will be appreciated by a person of ordinary skill in the art, mixing of the first and second precursor components can be enhanced by kneading package 10 after frangible seal 50 has been breached.

In one embodiment of a nitro-detection wipe, a first precursor component of the chemical detection solution comprises ethylenediamine dihydrochloride and a second precursor component of the chemical detection solution comprises sulfanilamide. In this embodiment, a packaged nitro-detection wipe includes a package having two compartments separated by a frangible seal, a first compartment containing a fibrous substrate impregnated with the first precursor component and a second compartment containing the second precursor component. In another embodiment of a nitro-detection wipe, a first precursor component of the chemical detection solution comprises dimethyl sulfoxide, an alcohol, tetrabutylammonium hydroxide, ethylenediamine dihydrochloride and, optionally, zinc and a second precursor component of the chemical detection solution comprises sulfanilamide and phosphoric acid. In this embodiment, a packaged nitro-detection wipe includes a package having two compartments separated by a frangible seal, a first compartment containing a fibrous substrate impregnated with the first precursor component and a second compartment containing the second precursor component.

In yet another embodiment of a nitro-detection wipe, a first precursor component of the chemical detection solution comprises dimethyl sulfoxide, tetrabutylammonium hydroxide, ethylenediamine dihydrochloride and isopropyl alcohol and a second precursor component of the chemical detection solution comprises sulfanilamide and phosphoric acid. In this embodiment, a packaged nitro-detection wipe includes a package having two compartments separated by a frangible seal, a first compartment containing a fibrous substrate impregnated with the first precursor component and a second compartment containing the second precursor component. In still another embodiment of a nitro-detection wipe, a first precursor component of the chemical detection solution comprises sulfanilamide and phosphoric acid and a second precursor component of the chemical detection solution comprises dimethyl sulfoxide, tetrabutylammonium hydroxide, ethylenediamine dihydrochloride and isopropyl alcohol. In this embodiment, a packaged nitro-detection wipe includes a package having two compartments separated by a frangible seal, a first compartment containing a fibrous substrate impregnated with the first precursor component and a second compartment containing the second precursor component. A person of ordinary skill in the art will appreciate that other combinations of these ingredients may alternatively be employed.

In one embodiment of a peroxide-detection wipe, a first precursor component of the chemical detection solution comprises dimethyl sulfoxide, isopropyl alcohol, diphenylamine, and sulfuric acid and a second precursor component of the chemical detection solution comprises dipropylene glycol dimethyl ether, ferric chloride, and hydrochloric acid. In this embodiment, a packaged peroxide-detection wipe includes a package having two compartments separated by a frangible seal, a first compartment containing a fibrous substrate impregnated with the first precursor component and a second compartment containing the second precursor component. In another embodiment of a peroxide-detection wipe, a first precursor component of the chemical detection solution comprises dipropylene glycol dimethyl ether, ferric chloride, and hydrochloric acid and a second precursor component of the chemical detection solution comprises dimethyl sulfoxide, isopropyl alcohol, diphenylamine, and sulfuric acid. In this embodiment, a packaged peroxide-detection wipe includes a package having two compartments separated by a frangible seal, a first compartment containing a fibrous substrate impregnated with the first precursor component and a second compartment containing the second precursor component. A person of ordinary skill in the art will appreciate that other combinations of ingredients may alternatively be employed.

In one embodiment of a fentanyl-wipe, a packaged fentanyl-detection wipe includes a package having a single compartment containing a fibrous substrate impregnated with a fentanyl detection formula comprised of an aqueous buffered solution of eosin Y of varying concentrations.

In one embodiment of a methamphetamine-wipe, a first precursor component of the chemical detection solution comprises an aqueous solution of sodium nitroprusside and acetaldehyde and a second precursor component of the chemical detection solution comprises an aqueous solution of sodium carbonate. In this embodiment, a packaged methamphetamine-wipe includes a package having two compartments separated by a frangible seal, a first compartment containing a fibrous substrate impregnated with the first precursor component and a second compartment containing the second precursor component. A person of ordinary skill in the art will appreciate that other combinations of ingredients may alternatively be employed.

In one embodiment of a cocaine-wipe, a packaged wipe includes a package having a single compartment containing a fibrous substrate impregnated with a cocaine detection formula comprised of cobalt thiocyanate, 10% acetic acid, glycerin and optionally one or more of hydrochloric acid and/or chloroform.

To make a packaged detection wipe in which precursor components of the chemical detection solution are kept separate in accordance with certain embodiments, a variety of manufacturing processes can be employed as would be envisioned by a person of ordinary skill in the art. In one example, a two-compartment packaged wipe product as depicted schematically in FIG. 2 can be made by folding a sheet of heat sealable laminated foil such that folded edge 111 forms a first side of package 110, heat sealing side 112 and side 113 of package 110 to form heat seals 115, 116 to form an envelope that is open on side 114 opposite fold edge 111 and that defines an internal chamber, forming frangible seal 150 to separate the chamber into first and second compartments 130, 140 by creating third heat seal 117 between side seals 112, 113 in an orientation generally perpendicular to folded edge 111, inserting individual towelette 120 into first compartment 130 thus formed, injecting a first precursor component of the chemical detection solution into contact with the towelette in first compartment 130 to impregnate towelette 120 with the first precursor component, injecting a second precursor component of the chemical detection solution into second compartment 140, and heat sealing side 114 to form heat seal 118, thereby providing sealed two-compartment package 110 containing towelette 120 and the first precursor component in first compartment 130 and the second precursor component in second compartment 140. If desired, heat seal 119 also can be formed at fold edge 111 before or after inserting towelette 120 and injecting the precursor components. For example, heat seal 119 can be formed at fold edge 111, if desired, simultaneously with heat sealing of the sides adjacent the fold.

Frangible seal 150 is weaker than heat seals 115, 116, 118, 119 of package 110. This can be achieved, for example, by controlling the time, temperature and pressure parameters used to form seals 115, 116, 118, 119 relative to seal 117. As will be appreciated by a person of ordinary skill in the art, a variety of heat sealable laminated foil materials are known in the art and readily available commercially. Such materials include a heat seal layer composed of a material that is operable to be fused to a heat seal layer of another piece of the heat sealable laminated foil material when the pieces are positioned such that the respective heat seal layer face one another upon application of heat and compressive force between the layers. Application of heat and pressure using opposing heater bars is effective to seal the respective heat seal layers to one another at the location between the heater bars. Moreover, the strength of such seal is a function of the temperature of the heater bars, the pressure applied by the heater bars and the length of time that the heat and pressure are applied. Therefore, by adjusting the time, temperature and pressure parameters, a frangible seal can be formed that is weaker than the edge seals of the package. For example, in one manner of making a two-compartment package as described herein, seals 115, 116, 118, 119 are formed using heater bars on each side that are set at a temperature of about 270° F., the heater bars applying a pressure of about 40 pounds per square inch (PSI) for a period of time to achieve sealing; and frangible seal 150 is formed using a heater bar on one side that is set at a temperature of about 360° F. and a cooler bar on the opposite side that is set at a temperature of about 68° F., the heater and cooler bars applying a pressure of about 40 PSI for a period of time to achieve formation of frangible seal 150. The fabrication actions described herein, and alternative fabrication actions that are in the purview of a skilled artisan, can be accomplished in an automated fashion using equipment that is set up to feed the heat sealable laminated foil material from storage rolls, complete folding, heat sealing, inserting and injecting actions as desired to form the two-compartment packaged product as described in a ready-to-use form. This disclosure also contemplates fabrication of packages having more than two compartments, which would be advantageously used for embodiments in which it is desired to separate reagents of a particular chemical detection solution into more than two precursor components. Such a package with more than two compartments can include more than one frangible seal and, in some embodiments, can include frangible seals with varying strengths.

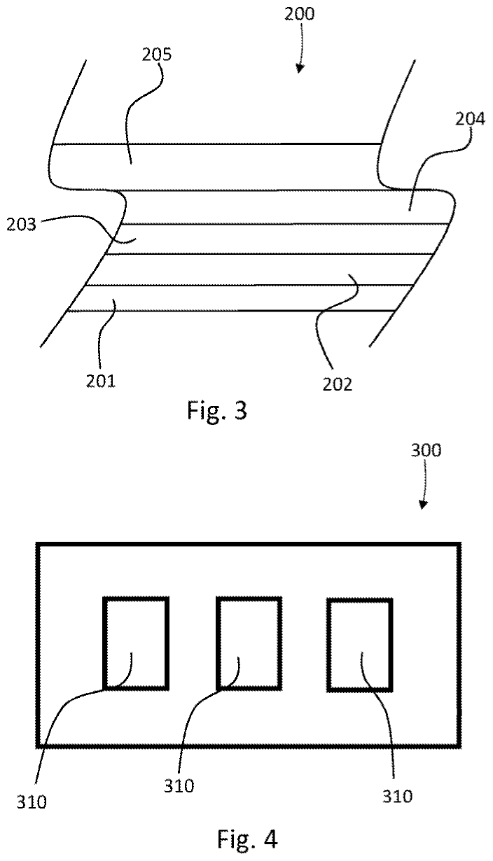

In one embodiment, depicted in FIG. 3, the heat sealable multilayer material used to make package 110 is a five-layer film. In one example, multilayer material 200 includes heat seal layer 201 composed of a heat seal material, first tie layer 202, barrier layer 203, second tie layer 204, and printable exterior layer 205. In one embodiment, barrier layer 203 is composed of a metallic foil. Printable exterior layer 205 in this embodiment is operable to be printed with information regarding the product contained within package 110. Foil barrier layer 203 is operable to prevent passage of oxygen gas and other gasses through multi-layer material 200, thereby isolating the contents of package 110 from environmental gases. Tie layers 202, 204 are operable to bind heat seal layer 201 to barrier layer 203 and to bind barrier layer 203 to printed exterior layer 205, respectively, in a manner whereby delamination of multilayer-material 200 is avoided under typical conditions of use. It is not intended that the present disclosure be limited to a 5-layer material as described above, it being understood that other materials, such as, for example, and without limitation, 7-layer laminate materials are also suitable for use in accordance with the present disclosure and are commercially available.

In another aspect of the disclosure, various methods for determining whether an explosive or illicit drug substance is present, whether in a bulk substance or in a trace amount on an object or surface, are contemplated. One such method includes providing a detection wipe fabricated from a fibrous substrate impregnated with a chemical detection solution in accordance with the present disclosure; wiping the object or surface with the detection wipe; observing whether the detection wipe exhibits a change in color; and determining whether the substance of interest is present on the surface based on whether the detection wipe exhibits a color change. The detection wipe can have a variety of characteristics and features in accordance with the embodiments described herein and contemplated by this disclosure. As will be explained in more detail below in connection with certain embodiments, if the user of a nitro-detection wipe determines that no color change has occurred, he or she can conclude that a nitro compound is not present on the surface or in the solid or liquid sample that has been tested. On the other hand, if a color change is observed by the user, then a determination can be made that a nitro compound has been positively identified as being present on the surface or in the solid or liquid sample. If the user of a hydrogen peroxide-detection wipe determines that no color change has occurred, he or she can conclude that hydrogen peroxide is not present on the surface or in the solid or liquid sample that has been tested. On the other hand, if a color change is observed by the user, then a determination can be made that hydrogen peroxide has been positively identified as being present on the surface or in the solid or liquid sample. If the user of a chlorate/perchlorate-detection wipe determines that no color change has occurred, he or she can conclude that a chlorate or perchlorate is not present on the surface or in the solid or liquid sample that has been tested. On the other hand, if a color change is observed by the user, then a determination can be made that a chlorate or perchlorate has been positively identified as being present on the surface or in the solid or liquid sample. If the user of a fentanyl-detection wipe determines that no color change has occurred, he or she can conclude that fentanyl containing compound is not present on the surface or in the solid or liquid sample that has been tested. On the other hand, if a color change is observed by the user, then a determination can be made that a fentanyl containing compound has been positively identified as being present on the surface or in the solid or liquid sample. If the user of a methamphetamine-detection wipe determines that no color change has occurred, he or she can conclude that a methamphetamine containing compound is not present on the surface or in the solid or liquid sample that has been evaluated. On the other hand, if a color change is observed by the user, then a determination can be made that a methamphetamine containing compound has been positively identified as being present on the surface or in the solid or liquid sample. If the user of a cocaine-detection wipe determines that no color change has occurred, he or she can conclude that cocaine containing compound is not present on the surface or in the solid or liquid sample that has been tested. On the other hand, if a color change is observed by the user, then a determination can be made that a cocaine containing compound has been positively identified as being present on the surface or in the solid or liquid sample.

Another method of determining whether an explosive or illicit drug is present on a surface or in the solid or liquid sample that is contemplated by this disclosure includes providing a detection wipe fabricated from a fibrous substrate; contacting the detection wipe with the surface or in the solid or liquid sample; then, after said contacting, introducing onto the wipe a chemical detection solution in accordance with the present disclosure; and determining whether the explosive or illicit drug substance is present on the surface or in the solid or liquid sample based on whether the detection wipe exhibits a color change. Each of the substrate and the chemical detection solution in this embodiment can have a wide variety of characteristics and features in accordance with the embodiments described herein.

Yet another method of determining whether an explosive or illicit drug substance is present on a surface or in the solid or liquid sample includes dispensing a detection wipe comprising a fibrous substrate from a container that contains multiple detection wipes and that is operable to dispense wipes individually; introducing onto the wipe a chemical detection solution in accordance with the present disclosure to provide an impregnated wipe; wiping the surface or contacting the solid or liquid sample with the impregnated detection wipe; observing whether the impregnated detection wipe exhibits a change in color; and determining whether the explosive or illicit drug substance is present on the surface or in the solid or liquid sample based on whether the impregnated detection wipe exhibits a color change. Yet another method of determining whether a target substance is present on a surface or in the solid or liquid sample includes dispensing a detection wipe comprising a fibrous substrate from a container that contains multiple detection wipes and that is operable to dispense wipes individually; wiping the surface or contacting the solid or liquid sample with the detection wipe; after said wiping or contacting, introducing onto the wipe a chemical detection solution in accordance with the present disclosure; observing whether the detection wipe exhibits a change in color; and determining whether an explosive or illicit drug substance is present on the surface or in the solid or liquid sample based on whether the detection wipe exhibits a color change.

Because the present disclosure includes diverse formulations for detecting the presence of diverse substances on a surface, it provides convenient methods for efficiently testing a surface or a solid or liquid sample for multiple substances of interest. For example, one embodiment of such a method of determining whether a substance of interest is present on a surface or in a solid or liquid sample includes providing a first detection wipe fabricated from a fibrous substrate; introducing a first chemical detection solution onto the first wipe, the first chemical detection solution including at least one reagent operable to undergo a chemical reaction or a series of chemical reactions to produce a compound having a visible color when the solution contacts a first target substance, which can be, for example, an explosive substance or an illicit drug; wiping the surface or contacting the solid or liquid sample with the first detection wipe; observing whether the first detection wipe exhibits a change in color; determining whether the first substance of interest is present on the surface or in the solid or liquid sample based on whether the first detection wipe exhibits a color change; providing a second detection wipe fabricated from a fibrous substrate; introducing a second chemical detection solution onto the second wipe, the second chemical detection solution including at least one reagent operable to undergo a chemical reaction or a series of chemical reactions to produce a compound having a visible color when the solution contacts a second substance of interest; wiping the surface or contacting the solid or liquid sample with the second detection wipe; observing whether the second detection wipe exhibits a change in color; and determining whether the second explosive or illicit drug substance is present on the surface or in the solid or liquid sample based on whether the second detection wipe exhibits a color change. In another embodiment, at least one of the first and second chemical detection solutions is a chlorate- or perchlorate-detection solution, a nitro-detection solution or a hydrogen peroxide-detection solution in the case of the explosives detection or a fentanyl-detection solution, a methamphetamine-detection solution or a cocaine-detection solution in the case of illicit drug screening, examples of which are described herein. In yet another embodiment, at least one of the first and second chemical detection solutions is selected from the group consisting of a chemical detection solution comprising dimethyl sulfoxide, an alcohol, an acid and diphenylamine; a chemical detection solution comprising Griess reagents; and a chemical detection solution formed from a first solution that includes dipropylene glycol dimethyl ether, ferric chloride and hydrochloric acid and a second solution that includes dimethyl sulfoxide, isopropyl alcohol, diphenylamine and sulfuric acid. In still another embodiment, each of the first and second chemical detection solutions is a chlorate- or perchlorate-detection solution, a nitro-detection solution or a hydrogen peroxide-detection solution. In other embodiments, at least one of the first and second chemical detection solutions is selected from a fentanyl-detection solution, a methamphetamine-detection solution and a cocaine detection solution, examples of which are described herein. In yet another embodiment, at least one of the first and second chemical detection solutions is selected from the group consisting of a chemical detection solution comprising eosin Y; a chemical detection solution formed from a first solution comprising an aqueous solution of sodium nitroprusside and acetaldehyde and a second solution comprising an aqueous solution of sodium carbonate; and a third chemical detection solution comprised of cobalt thiocyanate, ten percent acetic acid, glycerine and optionally one or more of hydrochloric acid and/or chloroform. In still another embodiment, each of the first and second chemical detection solutions is a fentanyl-detection solution, a methamphetamine-detection solution or a cocaine-detection solution.

In another embodiment, the method further includes providing a third detection wipe fabricated from a fibrous substrate; introducing a third chemical detection solution onto the third wipe, the third chemical detection solution including at least one reagent operable to undergo a chemical reaction or a series of chemical reactions to produce a compound having a visible color when the solution contacts a third substance of interest; wiping the surface or contacting the solid or liquid sample with the third detection wipe; observing whether the third detection wipe exhibits a change in color; determining whether the third substance of interest is present on the surface or in the solid or liquid sample based on whether the third detection wipe exhibits a color change. In another embodiment, at least two of the first, second and third chemical detection solutions is a chlorate- or perchlorate-detection solution, a nitro-detection solution or a hydrogen peroxide-detection solution, examples of which are described herein. In yet another embodiment, at least two of the first, second and third chemical detection solutions are selected from the group consisting of a chemical detection solution comprising dimethyl sulfoxide, an alcohol, an acid and diphenylamine; a chemical detection solution comprising Griess reagents; and a chemical detection solution comprising dimethyl sulfoxide, isopropyl alcohol, diphenylamine, sulfuric acid, dipropylene glycol dimethyl ether, ferric chloride and hydrochloric acid. In still another embodiment, each of the first, second and third chemical detection solutions is a chlorate- or perchlorate-detection solution, a nitro-detection solution or a hydrogen peroxide-detection solution. In other embodiments, at least two of the first, second and third chemical detection solutions are selected from a fentanyl-detection solution, a methamphetamine-detection solution and a cocaine detection solution, examples of which are described herein. In yet another embodiment, at least two of the first, second and third chemical detection solutions are selected from the group consisting of a chemical detection solution comprising eosin Y; a chemical detection solution formed from a first solution comprising an aqueous solution of sodium nitroprusside and acetaldehyde and a second solution comprising an aqueous solution of sodium carbonate; and a third chemical detection solution comprised of cobalt thiocyanate, ten percent acetic acid, glycerine and optionally one or more of hydrochloric acid and/or chloroform. In still another embodiment, each of the first, second and third chemical detection solutions is a fentanyl-detection solution, a methamphetamine-detection solution or a cocaine-detection solution.

Also provided by this disclosure are kits for detecting the presence of one or more explosive substances on a surface or in a solid or liquid sample, kits for detecting the presence of one or more illicit drug on a surface or in a solid or liquid sample, and kits suitable for detecting the presence or absence of both explosive substances and illicit drugs on a surface or in a solid or liquid sample. In one embodiment, a kit to test for a plurality of diverse explosive substances on a surface or in a solid or liquid sample includes a plurality of wipes, each of said plurality of wipes composed of a fibrous substrate; a plurality of containers; and a plurality of chemical detection solutions, each of said plurality of chemical detection solutions contained within one of said plurality of containers and including at least one reagent operable to undergo a chemical reaction or a series of chemical reactions to produce a compound having a visible color when the solution contacts an explosive substance. In another embodiment, the kit includes a first wipe composed of a first fibrous substrate and a first chemical detection solution impregnated into the first substrate, the first chemical detection solution including at least one reagent operable to undergo a chemical reaction or a series of chemical reactions to produce a compound having a visible color when the first solution contacts a first explosive substance, the first wipe being configured to be wiped over a surface or contacted with a solid or liquid sample for detection of the presence or absence of the first explosive substance on the surface or in the solid or liquid sample; and a second wipe composed of a second fibrous substrate and a second chemical detection solution impregnated into the second substrate, the second chemical detection solution including at least one reagent operable to undergo a chemical reaction or a series of chemical reactions to produce a compound having a visible color when the second solution contacts a second explosive substance, the second wipe being configured to be wiped over a surface or contacted with a solid or liquid sample for detection of the presence or absence of the second explosive substance on the surface or in the solid or liquid sample. In yet another embodiment, each of the first and second chemical detection solutions is selected from a chlorate- or perchlorate-detection solution, a nitro-detection solution and a hydrogen peroxide-detection solution. In still another embodiment, each of the first and second chemical detection solutions is selected from the group consisting of a chemical detection solution comprising dimethyl sulfoxide, an alcohol, an acid and diphenylamine; a chemical detection solution comprising Griess reagents; and a chemical detection solution formed from a first solution that includes dipropylene glycol dimethyl ether, ferric chloride and hydrochloric acid and a second solution that includes dimethyl sulfoxide, isopropyl alcohol, diphenylamine and sulfuric acid.

In another embodiment, the explosive detection kit further includes a third wipe composed of a third fibrous substrate and a third chemical detection solution impregnated into the third substrate, the third chemical detection solution including at least one reagent operable to undergo a chemical reaction or a series of chemical reactions to produce a compound having a visible color when the third solution contacts a third explosive substance, the third wipe being configured to be wiped over a surface or contacted with a solid or liquid sample for detection of the presence or absence of the third explosive substance on the surface or in the solid or liquid sample. In yet another embodiment, each of the first, second and third chemical detection solutions is selected from a chlorate- or perchlorate-detection solution, a nitro-detection solution and a hydrogen peroxide-detection solution. In still another embodiment, each of the first, second and third chemical detection solutions is selected from the group consisting of a chemical detection solution comprising dimethyl sulfoxide, an alcohol, an acid and diphenylamine; a chemical detection solution comprising methylene blue; a chemical detection solution comprising Griess reagents; and a chemical detection solution formed from a first solution that includes dipropylene glycol dimethyl ether, ferric chloride and hydrochloric acid and a second solution that includes dimethyl sulfoxide, isopropyl alcohol, diphenylamine and sulfuric acid.

Also provided by this disclosure are kits for detecting the presence of one or more illicit drug substances on a surface or in a solid or liquid sample. In one embodiment, a kit to test for a plurality of diverse illicit drug substances on a surface or in a solid or liquid sample includes a plurality of wipes, each of said plurality of wipes composed of a fibrous substrate; a plurality of containers; and a plurality of chemical detection solutions, each of said plurality of chemical detection solutions contained within one of said plurality of containers and including at least one reagent operable to undergo a chemical reaction or a series of chemical reactions to produce a compound having a visible color when the solution contacts an illicit drug substance. In another embodiment, the kit includes a first wipe composed of a first fibrous substrate and a first chemical detection solution impregnated into the first substrate, the first chemical detection solution including at least one reagent operable to undergo a chemical reaction or a series of chemical reactions to produce a compound having a visible color when the first solution contacts a first illicit drug substance, the first wipe being configured to be wiped over a surface or contacted with a solid or liquid sample for detection of the presence or absence of the first illicit drug substance on the surface or in the solid or liquid sample; and a second wipe composed of a second fibrous substrate and a second chemical detection solution impregnated into the second substrate, the second chemical detection solution including at least one reagent operable to undergo a chemical reaction or a series of chemical reactions to produce a compound having a visible color when the second solution contacts a second illicit drug substance, the second wipe being configured to be wiped over a surface or contacted with a solid or liquid sample for detection of the presence or absence of the second illicit drug substance on the surface or in the solid or liquid sample. In yet another embodiment, each of the first and second chemical detection solutions is selected from a fentanyl-detection solution, a methamphetamine-detection solution and a cocaine-detection solution. In still another embodiment, each of the first and second chemical detection solutions is selected from the group consisting of a chemical detection solution comprising eosin Y, a chemical detection solution formed from a first solution comprised of an aqueous solution of sodium nitroprusside and acetaldehyde and a second solution including an aqueous solution of sodium carbonate, and a chemical detection solution formed from cobalt thiocyanate, ten percent acetic acid, glycerine and optionally one or more of hydrochloric acid and/or chloroform.

In another embodiment, the illicit drug detection kit further includes a third wipe composed of a third fibrous substrate and a third chemical detection solution impregnated into the third substrate, the third chemical detection solution including at least one reagent operable to undergo a chemical reaction or a series of chemical reactions to produce a compound having a visible color when the third solution contacts a third illicit drug substance, the third wipe being configured to be wiped over a surface or contacted with a solid or liquid sample for detection of the presence or absence of the third illicit drug substance on the surface or in the solid or liquid sample. In yet another embodiment, each of the first, second and third chemical detection solutions is selected from a fentanyl-detection solution, a methamphetamine-detection solution and a cocaine-detection solution. In still another embodiment, each of the first, second and third chemical detection solutions is selected from the group consisting of a chemical detection solution comprising eosin Y, a chemical detection solution formed from a first solution comprised of an aqueous solution of sodium nitroprusside and acetaldehyde and a second solution including an aqueous solution of sodium carbonate, and a chemical detection solution formed from cobalt thiocyanate, ten percent acetic acid, glycerine and optionally one or more of hydrochloric acid and/or chloroform.

In another aspect of the disclosure, a kit is provided to test for a plurality of diverse explosive or illicit drug substances on a surface. In one embodiment, kit 300 depicted schematically in FIG. 4 includes a plurality of individual use detection wipe packages 310. For example, the plurality of individual use detection wipe packages for explosives detection can include at least one, at least two, at least three, at least four, at least five or at least six or more detection wipes selected from the group consisting of a chlorate- and/or perchlorate-detection wipe as disclosed herein, a hydrogen peroxide-detection wipe as disclosed herein, a nitro-detection wipe as disclosed herein, a fentanyl-detection wipe as disclosed herein, a methamphetamine-detection wipe as disclosed herein and a cocaine-detection wipe as disclosed herein. In another embodiment, the drug detection kit includes any number of a chlorate- and/or perchlorate-detection wipe as disclosed herein, a hydrogen peroxide-detection wipe as disclosed herein, a nitro-detection wipe as disclosed herein, a fentanyl-detection wipe as disclosed herein, a methamphetamine-detection wipe as disclosed herein, and a cocaine-detection wipe as disclosed herein.

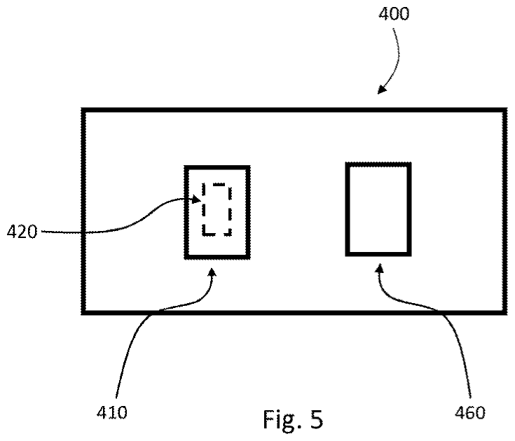

In another embodiment of a kit to test for a plurality of diverse explosive substances on a surface, kit 400 depicted schematically in FIG. 5 includes wipe 420 contained within a chamber of sealed package, wipe 410 impregnated with a first chemical detection solution comprising dimethyl sulfoxide, an alcohol, an acid and diphenylamine; wherein the first chemical detection solution is operable to produce a compound having a visible color when the solution contacts a chlorate. Kit 400 also includes container 460 and, contained within container 460, either an aqueous solution of methylene blue or a mixture comprising dipropylene glycol dimethyl ether, ferric chloride and hydrochloric acid. A mixture of the first chemical detection solution impregnated in wipe 410 (which alone is operable to produce a compound having a visible color when the solution contacts a chlorate), with a quantity of the mixture contained in container 460, enables use of the same wipe 410 to detect multiple explosive substances. More particularly, wipe 410 can first be used for detection of chlorate, then can be used for detection of perchlorate by delivering a quantity of a solution of methylene blue from container 460 onto wipe 410. Alternatively, wipe 410 can first be used for detection of chlorate, then can be used for detection of a peroxide by delivering a quantity of a solution comprising dimethyl sulfoxide, an alcohol, an acid and diphenylamine from container 460 onto wipe 410. In some embodiments, kit 400 includes a plurality of wipes impregnated with the first chemical detection solution and a plurality of containers 460, each containing either an aqueous solution of methylene blue or a mixture comprising dipropylene glycol dimethyl ether, ferric chloride and hydrochloric acid in a sufficient quantity for multiple uses with multiple wipes.

While this disclosure presents data for the creation and use of a variety of explosive detection wipes and drug wipes, the disclosures herein are also applicable to the creation of wipes for other applications, using other colorimetric reagents for other kinds of chemical discoveries and detections. For instance, the drug detection suite described in this application can be augmented by the creation of another wipe designed for the detection of artificial cannabinoid compounds or psychoactive substances such as K2, spice or LSD, using a small amount of Earlich's reagent contained within a sealed wipe.

For example, in another alternative embodiment, this disclosure contemplates of the use of Ehrlich reagent for determining whether an illicit substance is present on a surface. Ehrlich reagent is a reagent containing p-dimethylaminobenzaldehyde (DMAB), as shown below.

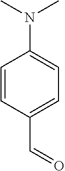

The Ehrlich reagent works by binding to the C2 position of two indole moieties to form a resonance stabilized carbenium ion compound. The electrophilic substitution reaction of indoles with carbonyl compounds resulting in the formation of bis(indolyl)alkanes in an acid-catalyzed reaction has been used to determine a color change from yellow to purple/blue.

The Ehrlich reagent is prepared by the addition of about 0.5 to about 2.0 grams of p-dimethylaminobenzaldehyde (DMAB) in 50 mL of 95% ethanol. To this, about 50 mL of 10% sulfuric acid is added. Other alcohols, such as 1-propanol, can also be used and other acids, such as hydrochloric acid can be used as well. The Ehrlich reagent is similar to a number of other indole tests, and thus acts as an indicator to identify indoles.

Ehrlich reagent wipes can be used for the detection of LSD and detection of synthetic cannabinoids, also sometimes called cannabimimetics (“synthetic marijuana,” “Spice,” “K2”) as they are operable to change color when the solution comes into contact with LSD or synthetic cannabinoids (if present). Typically, synthetic cannabinoids are sprayed onto plant matter and can be smoked, or ingested as a concentrated liquid They have been marketed as herbal incense, or “herbal smoking blends”, and sold under common names like K2, spice and synthetic marijuana. These chemicals are called cannabinoids because they are like chemicals found in the marijuana plant. Synthetic cannabinoids are unregulated mind-altering substances that have become newly available on the market and are intended to produce the same effects as marijuana. Some of these substances may have been around for years but have reentered the market in altered chemical forms, or due to renewed popularity. There a several analogs that are available, including but not limited to benzoylindoles (HU-210, AM-694, RCS-4, WIN 48,098), Naphthoylindoles, (JWH-018, JWH-019, JWH-073, JWH-081, JWH-122, JWH-210, AM-2201, and Phenylacetylindole (JWH-250, JWH-250, RCS-8), as shown below.

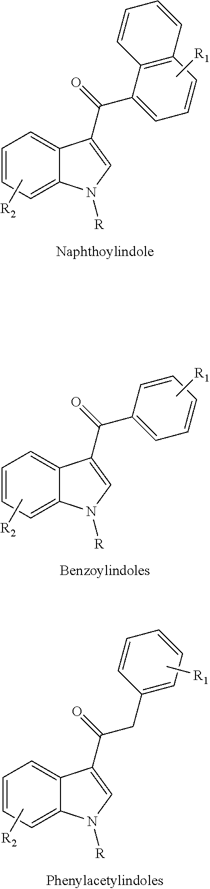

As with other wipe embodiments described herein, an Ehrlich reagent wipe can be contained within a sealed package that can be opened and used for drug detection by wiping a surface or contacting a solid or liquid sample with the impregnated detection wipe, observing whether the impregnated detection wipe exhibits a change in color and determining whether the illicit drug containing psychoactive substances such as K2, spice or LSD, is present based on whether the impregnated detection wipe exhibits a color change. This wipe can be used as a part of a kit with other drug discovery wipes as mentioned above. Other indole tests are also contemplated by this disclosure for detection of LSD and/or synthetic cannabinoids, including, for example and without limitation, the van Urk reagent, which uses 0.125 g of p-DMAB, 0.2 mL of ferric chloride solution (25 g/mL) in 100 mL of 65% sulfuric acid (sometimes referred to as the Hofmann reagent or p-DMAB-TS (Test Solution)) and gives slightly different colors with different indoles; the Renz and Loew reagent, which uses p-dimethylaminocinnamaldehyde and may also be used for the detection of flavonoids; and the “improved hallucinogen reagent,” which uses a 1:1 solution of 5% DMAB in concentrated phosphoric acid (specific gravity 1.45) to methanol.

Various processes, methods, compositions, and devices of the present disclosure are further demonstrated in the following examples. These examples are illustrative only and are not intended to limit or preclude other embodiments of the present invention.

### Example 1: Preparation and Use of a Hydrogen Peroxide Detection Wipe

An illustrative absorbent nonwoven substrate wipe for detecting a peroxide based explosive material was fabricated as follows:
A first solution was prepared by mixing 7.4% dimethyl sulfoxide, 6.0% isopropyl alcohol, 0.5% diphenylamine and 86.0% sulfuric acid (50% solution), all by weight.
The first solution was then combined at a 1:1 ratio, by volume, with a second solution made by mixing 98.4% dipropylene glycol dimethyl ether, 0.8% ferric chloride and 0.073% hydrochloric acid (30% solution), all by weight to provide a chemical detection solution. When packaged, the first solution is contained in 410 as demonstrated in FIG. 5, while the second solution is contained within 460.
A hydroentangled and calendared nonwoven absorbent wipe obtained from Suominen Nonwovens (Bethune, South Carolina) was impregnated with the final solution.

After fabrication and activation by combining the two solutions by bursting the frangible seal, the wipe was contacted with hydrogen peroxide (also referred to herein as “peroxide”), which is a known explosive substance when in the concentrated form (i.e., is used to make certain explosives). Upon contact with the peroxide, the wipe quickly turned to an intense blue violet coloration, indicating the presence of the hydrogen peroxide.

In the presence of the peroxide, the Fe3+ species in the chemical detection solution catalyzed the decomposition of the peroxide, thereby producing, among other things, reduced Fe2+, while the Fe2+ further reacted with the peroxide and oxidized to Fe3+. In the presence of the redox indicator diphenylamine, the redox process turned the color indicator from clear (oxidized form) to color (reduced form). The end point was marked with an intense blue violet coloration. The reduction potential value of the system was E° red=+0.76V, which was noted as being near to the ferrous-ferric system (E° red=+0.77V).

Regarding the chemistry behind the color change, since the wipe contained Fe3+, sulfuric acid, hydrochloric acid, and diphenylamine, when the wipe was exposed to the material containing H2O2, the Fe3+ catalyzed the decomposition of the H2O2 into H2O. As a result, it reduced Fe3+ to Fe2+(among other species), which in this acidic medium and in the presence of H2O2, generated a Fenton's reagent. As is shown within the chemical reactions listed below, those of skill in the art will understand and appreciate that a Fenton's reagent is a solution that is formed by reaction of Fe2+ and hydrogen peroxide. That is, Fe2+ is oxidized to Fe3+ by hydrogen peroxide to form a hydroxyl radical and a hydroxyl anion. Fe3+ is then reduced back to Fe2+ by the same hydrogen peroxide to a peroxide radical and a proton.

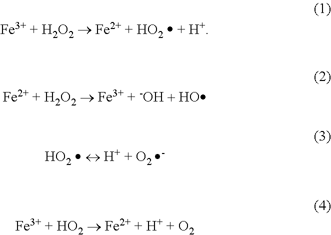

The diphenylamine in the wipe operates as the redox color indicating agent that is responsible for generating the color species that resulted in the observed color change. The chemical reaction that took place is shown below. As those of skill in the art will understand and appreciate, the action of diphenylamine (I) as a redox color indicator depends upon its oxidation first into colorless diphenylbenzidine (II), which becomes the operative indicator in the solution and is reversibly further oxidized to diphenylbenzidine violet (III).

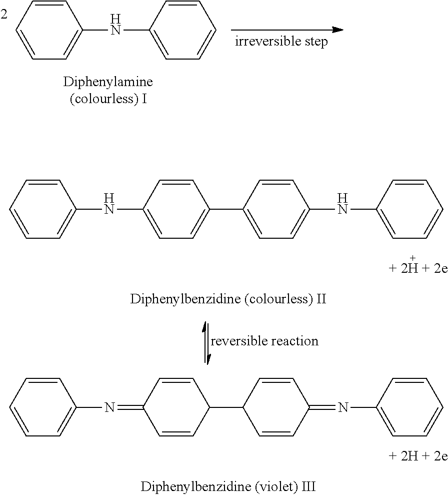 [SMILES: c1ccc(Nc2ccccc2)cc1 ; c1ccc(Nc2ccc(-c3ccc(Nc4ccccc4)cc3)cc2)cc1 ; C1=CC(C2C=CC(=Nc3ccccc3)C=C2)C=CC1=Nc1ccccc1]

### Example 2: Double Blind Study of Hydrogen Peroxide Detection Wipes

A double-blind test was performed to evaluate the accuracy with which hydrogen peroxide based explosive detection wipes made as described in Example 1 identify the presence of hydrogen peroxide on test surfaces.

### Set-Up

Thirty (30) human test subjects were recruited to conduct this study. The experiment was performed in a controlled setting, isolated from laboratories or other means of contamination. Numbered stickers, numbered from one (1) to thirty (30) were prepared to ensure that the test subjects were kept in order.

Several sets of thirty (30) test cards were prepared so that multiple trials could be run in the same session. The test cards consisted of a piece of construction paper with a smaller, white card in the middle, the latter having been contacted with a peroxide or nitrate-containing substance or nothing at all.

### Procedure

The thirty (30) subjects were randomly divided into three (3) groups of ten (10) subjects, referred to herein as “primary groups.” Each primary group was dubbed “A,” “B,” or “C.” Each subject was given a test card and instructed to not touch the smaller, white card in the center as doing so could skew the results of the test. Each subject also was given a primary group card with either “A,” “B,” or “C” written on it, depending upon the primary group the subject was randomly placed in, and each subject was instructed to keep the primary group cards concealed until the final station.

Subjects in primary group A were given cards that had been in contact with a nitrate-containing substance. Subjects in primary group B received cards that had not come into contact with any substance. Subjects in primary group C receive cards that had come into contact with peroxide-containing compounds. Neither the subjects nor the persons recording results (test operators) were informed which primary group had which type of sample. The only person who knew which card the subjects received was the person who passed them out, who had no further role in the double-blind study.

Out of the thirty (30) subjects a random number generator was used to form a first group of ten (10), referred to herein as a “secondary group,” which was a random mixture of subjects from all three primary groups. A test operator at a first station assigned an order to the ten (10) subjects in the secondary group and dispensed a numbered sticker to each test subject so that the exact order was maintained within the secondary group throughout the trial. This process was then repeated using the random number generator to form a second secondary group of ten (10) subjects from the remaining twenty (20) subjects and was again repeated with the remaining ten (10) subjects, who formed a third secondary group, thereby randomly generating three secondary groups.

The first secondary group of ten (10) subjects then proceeded to the second station, one at a time in chronological order, where another test operator tested the subject's card by wiping a hydrogen peroxide based explosive detection wipe made in accordance with Example 1 over the card. Yet another test operator logged the results on data sheets, the result from each card tested being a positive or a negative result. A positive result was logged for a given card, indicating that the card was determined to contain peroxide, if the swiped area turned blue violet after the card was wiped. A negative result was logged for a given card, indicating that the card was determined to not contain peroxide if the swiped area did not change color after the card was wiped. Once results were logged for a given card, the card was discarded.

The subject then proceeded to the third and final station, where another test operator logged the subject's order and group information. At this station, the subject revealed his or her primary group card to the test operator, which information also was recorded by the test operator. The subject then re-concealed his or her primary group card, returned to his or her respective secondary group, received a new test card, and waited to be called again.

After a sufficient amount of data (more than 200 test points) was acquired, testing ended. Data from the testing station and group station were compared by an independent third party, and the data was analyzed to measure the accuracy of the hydrogen peroxide based explosive detection wipes to accurately indicate whether a hydrogen peroxide residue was present on the respective test cards.

Sample Data

| | |
|---|---|
| Sample of Data | Sample of Data |
| Collected at Testing Station | Collected at Group Station |

| Subject | Peroxide | Subject | Group |
|---|---|---|---|
| 1 | + | 1 | C |
| 2 | − | 2 | A |
| 3 | + | 3 | C |
| 4 | − | 4 | B |
| 5 | − | 5 | A |

The sample data displayed above demonstrates how the two data sets collected as described above were used to verify whether results logged at the testing station were accurate by comparing them to the group station data. For example, in the above sample data, subjects 1 and 3 tested positive for peroxide and were from Group C, which would be a correct result. Subjects 2 and 5, which were from Group A, and subject 4, which was from Group B, tested negative for peroxide, which also would be correct results.

To quantify the results, each correct result was given a value of 100 and each incorrect result was given a value of 0. These values were then averaged once all the results were determined. This average represents the percent accuracy of the hydrogen peroxide based explosive detection wipes.

### Results

The results of the double-blind test described above showed that the detection wipes tested were 100% accurate in identifying the presence of hydrogen peroxide with no false positives.

### Example 3: Preparation and Use of a Nitro Detection Wipe

An illustrative absorbent nonwoven substrate wipe for detecting a nitrogen based explosive material was fabricated as follows:
A chemical detection solution was prepared by first mixing dimethyl sulfoxide, methanol, tetrabutylammonium hydroxide, sulfanilamide, ethylenediamine dihydrochloride and phosphoric acid to provide a chemical detection solution having these ingredients in the following proportions, by weight: about 74% dimethyl sulfoxide, 9% methanol, 3% tetrabutylammonium hydroxide, 6% sulfanilamide, 0.2% ethylenediamine dihydrochloride and 8% phosphoric acid having a Molarity of about 85%. This first solution was combined in a 1:1 ratio with a second chemical solution of 0.88% zinc powder dissolved in butanol.
A hydroentangled and calendared nonwoven absorbent wipe obtained from Suominen Nonwovens (Bethune, South Carolina) was impregnated with the solution described in the preceding bullet. A second solution of sulfanilamide and 8% phosphoric acid was prepared in deionized water and sealed in the second compartment of a two-chambered package as shown in FIG. 2.

After fabrication, the wipe was activated by breaking the frangible seal and mixing the solutions in the two compartment of the package. The package was then opened and the wipe was contacted with a nitrate-containing substance, which is known to be present in “gunshot residue.” Upon contact with the nitrate-containing substance, the wipe quickly turned to an intense magenta color, indicating the presence of nitrite ions or nitro functional groups.

Generation of the color species that resulted in the observed color change occurred by a series of chemical reactions like the Griess reaction shown below.

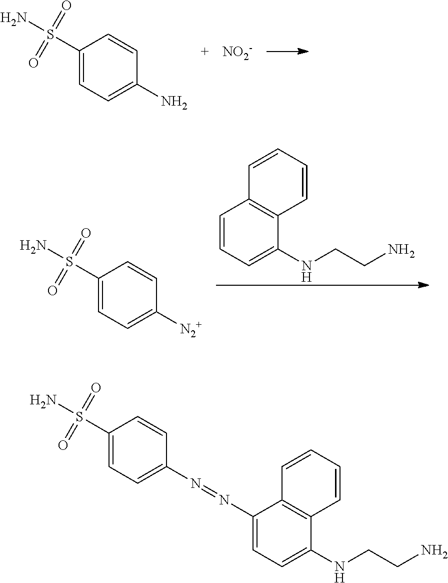

In this reaction, the sulfanimide reacts with a nitrite ion donated by the nitrate-containing substance to produce a diazonium salt. The diazonium salt then reacted with ethylenediamine dihydrochloride (represented in the Griess reaction shown above as N-(1-Naphthyl)ethylenediamine) to produce an azo dye (represented the Griess reaction shown above as N-alpha-naphthyl-ethylenediamine), which produces the magenta color.

### Example 4: Double Blind Study of Nitro Detection Wipes

A double-blind test was performed to evaluate the accuracy with which nitro detection wipes made as described in Example 3 identify the presence of nitrogen-based explosives on test surfaces. The Set-Up and Procedure used for this double-blind test is the same as the Set-Up and Procedure described in Example 2 except for the following differences:

The test operator at the second station tested the subject's card by wiping a nitro detection wipe made in accordance with Example 3 over the card. A positive result was logged by another test operator for a given card, indicating that the card was determined to contain a nitrate-containing substance, if the swiped area turned magenta after the card was wiped. A negative result was logged for a given card, indicating that the card was determined to not contain a nitrate-containing substance if the swiped area did not change color after the card was wiped.

After enough data (more than 200 test points) was acquired, testing ended. Data from the testing station and group station were compared by an independent third party, and the data was analyzed to measure the accuracy of the nitrogen based explosive detection wipes to accurately indicate whether a nitrate-containing residue was present on the respective test cards.

Sample Data

| | |
|---|---|
| Sample of Data | Sample of Data |
| Collected at Testing Station | Collected at Group Station |

| Subject | Nitrate | Subject | Group |
|---|---|---|---|
| 1 | + | 1 | C |
| 2 | − | 2 | A |
| 3 | + | 3 | C |
| 4 | − | 4 | B |
| 5 | − | 5 | A |

The sample data displayed above demonstrates how the two data sets collected as described above were used to verify whether results logged at the testing station were accurate by comparing them to the group station data. For example, in the above sample data, subjects 1 and 3 tested positive for a nitrate-containing substance and were from Group C, which would be a correct result. Subjects 2 and 5, which were from Group A, and subject 4, which was from Group B, tested negative for a nitrate-containing substance, which also would be correct results.

### Results

The results of the double-blind test described above showed that the nitro detection wipes tested were greater than 99% accurate in identifying the presence of a nitro- or nitrate-containing substance.

### Example 5: Preparation and Use of a Chlorate/Perchlorate Detection Wipe

An illustrative absorbent nonwoven substrate wipe for detecting a perchlorate/perchlorate based explosive material was fabricated as follows:
A first solution is prepared by mixing 7.4% dimethyl sulfoxide, 5.83% isopropyl alcohol, 0.47% diphenylamine and 86.29% sulfuric acid (30% solution), all by weight to provide a first solution.
The second solution is prepared containing methylene blue.
A hydroentangled and calendared nonwoven absorbent wipe obtained from Suominen Nonwovens (Bethune, South Carolina) was impregnated with the first solution and positioned in the first compartment of a two-chamber package such as that shown in FIG. 2, and the second solution was positioned in the second compartment of the two-chamber package, separated from the first solution by a frangible seal. In another embodiment, the first solution is contained within one compartment of a sealed wipe package while the second solution is contained within a second compartment of a sealed wipe package. The solutions are not mixed, but the second solution is allowed to flow onto the wipe without mixing or kneading the mixture to create a wipe with two solutions for dual detection of perchlorate and perchlorate.

After fabrication, the wipe is contacted with a chlorate or perchlorate, both known to be an explosive substance (i.e., is used to make certain explosives). Upon contact with the chlorate, the wipe quickly turned to a light blue coloration, indicating the presence of the chlorate. Upon contact with perchlorate, the wipe turns violet.

### Example 6: Preparation and Use of a Fentanyl-Detection Wipe

An illustrative absorbent nonwoven substrate wipe for detecting fentanyl is fabricated as follows:
A solution is prepared by dissolving eosin Y in water within a concentration range of from about 0.4% to about 2%, buffered with phosphate buffer ranging in pH from about 4 to about 9, and with yellow food coloring added to enhance the color observation.
A hydroentangled and calendared nonwoven absorbent wipe obtained from Suominen Nonwovens (Bethune, South Carolina) is impregnated with the chemical detection solution.

After fabrication, the wipe is contacted with fentanyl, a known illicit drug. Eosin Y binds to select tertiary amines to produce an orange to pink color change. More particularly, eosin Y binds to the nonpiperidine ring nitrogen of fentanyl as a primary binding site and the piperidine ring nitrogen as a secondary binding site.

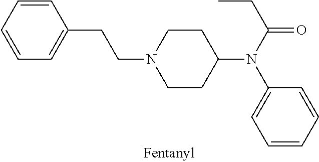 [SMILES: CCC(=O)N(c1ccccc1)C1CCN(CCc2ccccc2)CC1]

Upon contact with the fentanyl the wipe quickly turned from an orange color to a darker orange pink salmon color. This color change is visibly enhanced by shining UV B light on the wipe. Experimental research revealed that this wipe is operable to detect Fentanyl and fentanyl analogs. The wipe was tested against commercially available drugs designed as either an analytical standard material, or for pharmaceutical use, and was presented as either a powder or a liquid suspension as shown in the below table. In the case of powder, the wipe was touched to a small quantity of the powder, while in the case of a liquid presentation, the wipe was touched to the liquid.

| | Source | form presented | | | |
|---|---|---|---|---|---|
| | (pharmaceutical | (solid or liquid | | | |
| Drug Name | manufacturer) | patch) | concentration | | Result |
| Fentanyl | Mallinckrodt Co. | Liquid Patch | 25 | mcg/hr | Detected |
| Fentanyl Citrate | Sigma & Cerilliant | Liquid | 1 | mg/mL | Detected |
| Benzyl Fentanyl | SigSci | Liquid | 1 | mg/mL | Detected |
| Meperidine | Roxane Labs. | Solid | 50 | mg | Detected |
| Tramadol | Zydus Labs. | Solid | 50 | mg | Detected |
| Hydrocodone | Rhodes Pharmacy | Solid | 10 | mg | Detected |
| Naloxone | Narcan | Liquid | 4 | mg | Detected |
| Dimenhydrinate | Dramamine Inc. | Solid | 50 | mg | Detected |
| (Dramamine) | | | | | |

### Example 7: Preparation and Use of a Methamphetamine-Detection Wipe

An illustrative absorbent nonwoven substrate wipe for detecting a based methamphetamine containing material was fabricated as follows:
A first solution was prepared by mixing ethanol and acetaldehyde.
A second solution made by mixing an aqueous solution containing 1% sodium nitroprusside and 1% sodium carbonate by weight to provide a chemical detection solution.
A hydroentangled and calendared nonwoven absorbent wipe obtained from Suominen Nonwovens (Bethune, South Carolina) was impregnated with either the first or the second solution. The impregnated wipe was positioned in the first compartment of a two-chamber package such as that shown in FIG. 2, and the other solution was positioned in the second compartment of the two-chamber package, separated from the first solution by a frangible seal.

After fabrication, the frangible seal between the two compartments is broken, and the first and second solutions are mixed and adsorbed into the wipe prior to sampling. The internal frangible seam requires deliberate pressure to burst the interior weld between the two components, allowing for mix-in-the-bag activation. Contents are kept separate until just prior to when the wipe is to be used, allowing individually packaged components to remain inert and stable until being mixed and used. After activation, the wipe was contacted with methamphetamine containing substance. After contact, the wipe quickly turned from a neutral color to a cobalt blue coloration, indicating the presence of the methamphetamine.

### Example 8: Preparation and Use of a Cocaine-Detection Wipe

The cobalt thiocyanate test (or Scott's test) is a proven screening test for the presence of cocaine. An illustrative absorbent nonwoven substrate wipe for detecting cocaine was fabricated as follows:
A solution was prepared by dissolving 1% cobalt thiocyanate in a solution comprised of half 10% acetic acid and half glycerin. To this, another 6% by volume of chloroform was added. The resultant complex created a blue color.

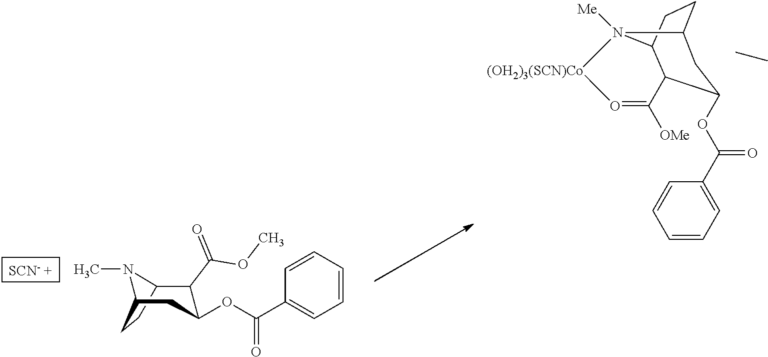

A hydroentangled and calendared nonwoven absorbent wipe obtained from Suominen Nonwovens (Bethune, South Carolina) was impregnated with the chemical detection solution.
   The wipe was contacted with substance containing cocaine: the wipe quickly turned from a neutral color to a sky-blue color.

As will be appreciated from a careful reading of the descriptions in the present disclosure, a wide variety of aspects and embodiments are contemplated by the present disclosure, examples of which include, without limitation, the aspects and embodiments listed below:

A packaged chemical detection wipe operable to detect the presence of an explosive substance or an illicit drug substance that includes: (i) a flexible package including a first seal surrounding a first compartment and a second compartment and including a second seal between the first compartment and the second compartment, (ii) a fibrous substrate positioned in the first compartment; (iii) a first precursor component of a chemical detection solution positioned in the first compartment and impregnated within the fibrous substrate; and (iv) a second precursor component of the chemical detection solution positioned in the second compartment; wherein mixture of the first precursor component and the second precursor component produces a chemical detection solution operable, when contacted with the explosive substance or the illicit drug substance, to undergo a chemical reaction or a series of chemical reactions to produce a compound having a characteristic visible color.

A packaged chemical detection wipe in accordance with any other embodiment described herein wherein the second seal is a frangible seal configured to be breached upon application to the first compartment or the second compartment a compressive force of a magnitude sufficient to breach the frangible seal but insufficient to breach the first seal, thereby permitting the first precursor component and the second precursor component to mix.

A packaged chemical detection wipe in accordance with any other embodiment described herein wherein the chemical detection wipe comprises a nitro-detection wipe; wherein the first precursor component comprises dimethyl sulfoxide, tetrabutylammonium hydroxide, ethylenediamine dihydrochloride and isopropyl alcohol; and wherein the second precursor component comprises sulfanilamide and phosphoric acid.

A packaged chemical detection wipe in accordance with any other embodiment described herein wherein the chemical detection wipe comprises a methamphetamine-detection wipe; wherein the first precursor component comprises an aqueous solution of sodium nitroprusside and acetaldehyde; and wherein the second precursor component comprises an aqueous solution of sodium carbonate.

A packaged chemical detection wipe in accordance with any other embodiment described herein wherein the chemical detection wipe comprises a peroxide-detection wipe; wherein the first precursor component comprises dipropylene glycol dimethyl ether, ferric chloride, and hydrochloric acid; and wherein the second precursor component comprises dimethyl sulfoxide, isopropyl alcohol, diphenylamine, and sulfuric acid.

A packaged chemical detection wipe in accordance with any other embodiment described herein wherein the second precursor component has a pH of from about 1.0 to about 6.0 or from about 2.5 to about 5.0.

A packaged chemical detection wipe in accordance with any other embodiment described herein wherein the second precursor component comprises from about 6 to about 9% dimethyl sulfoxide (by weight), from about 4 to about 8% isopropyl alcohol (by weight), from about 0.1 to about 2% diphenylamine (by weight) and from about 80% to about 90.0% by weight of sulfuric acid.

A packaged chemical detection wipe in accordance with any other embodiment described herein wherein the second precursor component comprises about 7.4% dimethyl sulfoxide (by weight), about 6.0% isopropyl alcohol (by weight), about 0.5% diphenylamine (by weight) and about 86.0% by weight of sulfuric acid.

A packaged chemical detection wipe in accordance with any other embodiment described herein wherein the sulfuric acid has a concentration of from about 1.5M to about 11.0M or from about 5.4M to about 5.8M.

A packaged chemical detection wipe in accordance with any other embodiment described herein wherein the sulfuric acid has a concentration of about 2.55M or about 5.6M.

A packaged chemical detection wipe operable to detect the presence of a chlorate explosive substance and a perchlorate explosive substance that includes: (i) a flexible package including a first seal surrounding a first compartment and a second compartment and including a second seal between the first compartment and the second compartment; (ii) a fibrous substrate positioned in the first compartment; (iii) a first chemical detection solution positioned in the first compartment and impregnated within the fibrous substrate; and (iv) a second chemical detection solution positioned in the second compartment; wherein the first chemical detection solution is operable, when contacted with a chlorate compound, to undergo a chemical reaction or a series of chemical reactions to produce a compound having a characteristic visible color; and wherein the second chemical detection solution is operable, when contacted with a perchlorate compound, to undergo a chemical reaction or a series of chemical reactions to produce a compound having a characteristic visible color.

A packaged chemical detection wipe in accordance with any other embodiment described herein wherein the second seal is a frangible seal configured to be breached upon application to the first compartment or the second compartment a compressive force of a magnitude sufficient to breach the frangible seal but insufficient to breach the first seal, thereby permitting the second chemical detection solution to wet a portion of the wipe.

A packaged chemical detection wipe in accordance with any other embodiment described herein wherein the first chemical detection solution comprises dimethyl sulfoxide, isopropyl alcohol, sulfuric acid and diphenylamine; and wherein the second chemical detection solution comprises an aqueous solution of methylene blue.

A packaged chemical detection wipe in accordance with any other embodiment described herein wherein the second chemical detection solution comprises an aqueous solution of methylene blue having a concentration of from about 0.1% to about 1.0%, a concentration of from about 0.2% to about 0.5% or a concentration of about 0.25%.

A packaged chemical detection wipe operable to detect the presence of fentanyl that includes: (i) a flexible package including a seal surrounding a compartment; (ii) a fibrous substrate positioned in the compartment; and (iii) a fluid chemical detection solution positioned in the compartment and impregnated into the substrate, the chemical detection solution operable to undergo a chemical reaction or a series of chemical reactions to produce a compound having a characteristic visible color when the solution contacts fentanyl; wherein the chemical detection solution comprises an aqueous buffered solution of eosin Y and optionally further comprises yellow food coloring.

A packaged chemical detection wipe operable to detect the presence of cocaine that includes: (i) a flexible package including a seal surrounding a compartment; (ii) a fibrous substrate positioned in the compartment; and (iii) a fluid chemical detection solution positioned in the compartment and impregnated into the substrate, the chemical detection solution operable to undergo a chemical reaction or a series of chemical reactions to produce a compound having a characteristic visible color when the solution contacts cocaine; wherein the chemical detection solution comprises cobalt thiocyanate, 10% acetic acid, glycerin and optionally one or both of hydrochloric acid and chloroform.

A packaged chemical detection wipe operable to detect the presence of an illicit substance that includes: (i) a flexible package including a seal surrounding a compartment; (ii) a fibrous substrate positioned in the compartment; and (iii) a fluid chemical detection solution positioned in the compartment and impregnated into the substrate, the chemical detection solution operable to undergo a chemical reaction or a series of chemical reactions to produce a compound having a characteristic visible color when the solution contacts the illicit substance; wherein the chemical detection solution comprises Ehrlich reagent.

A packaged chemical detection wipe in accordance with any other embodiment described herein wherein the Ehrlich reagent comprises p-dimethylaminobenzaldehyde (DMAB) dissolved in a mixture of an alcohol and an acid.

A packaged chemical detection wipe in accordance with any other embodiment described herein wherein the Ehrlich reagent is prepared by dissolving from about 0.5 to about 2.0 grams of DMAB in a solution of about 50 mL of 95% ethanol and about 50 mL of concentrated hydrochloric acid.

A packaged chemical detection wipe in accordance with any other embodiment described herein wherein the illicit drug comprises a psychoactive substances such as K2, spice or LSD.

A packaged chemical detection wipe in accordance with any other embodiment described herein wherein the fibrous substrate comprises a plurality of fibers selected from the group consisting of cellulose, polyamides, polyesters, polyethylenes, polypropylenes, polyacrylics, cellulose acetate, polylactic acid, silk, wool, glass, polyaramids, and combinations thereof.

A packaged chemical detection wipe in accordance with any other embodiment described herein wherein the fibrous substrate is a nonwoven material having multiple layers formed from fibers selected from the group consisting of polyolefin, polypropylene, polyethylene, ethylene copolymers and propylene copolymers.

A packaged chemical detection wipe in accordance with any other embodiment described herein wherein the fibrous substrate is an absorbent nonwoven material formed from a blend of spunbonded fibers.

A packaged chemical detection wipe in accordance with any other embodiment described herein wherein the blend of spunbonded fibers is selected from the group consisting of polypropylene, polyester and wood pulp.

A method of fabricating a detection system for cocaine, the method comprising: (i) providing a chemical detection solution that includes cobalt thiocyanate, 10% acetic acid, glycerin, and optionally one or both of hydrochloric acid and chloroform; (ii) providing a fibrous substrate; and (iii) impregnating the fibrous substrate with the chemical detection solution to provide an impregnated substrate.

A method in accordance with any other embodiment described herein wherein the impregnated substrate is configured to be wiped over a surface or touched to a solid or liquid sample for detection of the presence or absence of an illicit substance on the surface or in the sample.

A method in accordance with any other embodiment described herein, further comprising placing the impregnated substrate in a sealed package.

A method in accordance with any other embodiment described herein wherein the impregnated substrate is configured to be removed from the sealed package and wiped over a surface or touched to a solid or liquid sample for detection of the presence or absence of cocaine on the surface or in the sample.

A method of fabricating chemical detection wipe operable to detect an explosive substance or an illicit drug, the method comprising: (i) providing a chemical detection solution selected from the group consisting of: (a) a combination of dimethyl sulfoxide, an alcohol, an acid and diphenylamine; (b) a combination of dimethyl sulfoxide, isopropyl alcohol, sulfuric acid, diphenylamine, dipropylene glycol dimethyl ether, ferric chloride and hydrochloric acid; (c) a combination of ethylene diamine dihydrochloride, dimethyl sulfoxide, tetrabutylammonium hydroxide, isopropyl alcohol, sulfanilamide, butanol, zinc powder and phosphoric acid; (d) a combination of eosin Y reagent dissolved in phosphate buffered and yellow food coloring; (e) a combination of acetaldehyde, sodium nitroprusside and sodium carbonate; (f) a combination of cobalt thiocyanate, glycerin, an acid and optionally chloroform; and (g) a combination of p-dimethylaminobenzaldehyde (DMAB), an alcohol and an acid; (ii) providing a fibrous substrate; and (iii) impregnating the fibrous substrate with the chemical detection solution to provide an impregnated substrate.

A method in accordance with any other embodiment described herein wherein the impregnated substrate is configured to be wiped over a surface or contacted with a solid or liquid sample for detection of the presence or absence of an explosive substance or an illicit drug on the surface or in the solid or liquid sample.

A method in accordance with any other embodiment described herein wherein the plurality of compounds and the substrate are contained within a sealed package.

A method in accordance with any other embodiment described herein, further comprising placing the impregnated substrate in a sealed package.

A method in accordance with any other embodiment described herein wherein the impregnated substrate is configured to be removed from the sealed package and wiped over a surface or contacted with a solid or liquid sample for detection of the presence or absence of an explosive substance or an illicit drug on the surface or in the solid or liquid sample.

A method of determining whether a target explosive or illicit drug substance is present on a surface or in a solid or liquid sample, comprising: (i) providing a detection wipe fabricated from a fibrous substrate impregnated with a chemical detection solution, the chemical detection solution selected from the group consisting of: (a) a combination of dimethyl sulfoxide, an alcohol, an acid and diphenylamine; (b) a combination of dimethyl sulfoxide, isopropyl alcohol, sulfuric acid, diphenylamine, dipropylene glycol dimethyl ether, ferric chloride and hydrochloric acid; (c) a combination of ethylene diamine dihydrochloride, dimethyl sulfoxide, tetrabutylammonium hydroxide, isopropyl alcohol, sulfanilamide, butanol, zinc powder and phosphoric acid; (d) a combination of eosin Y reagent dissolved in phosphate buffered and yellow food coloring; (e) a combination of acetaldehyde, sodium nitroprusside and sodium carbonate; (f) a combination of cobalt thiocyanate, glycerin, an acid and optionally chloroform; and (g) a combination of p-dimethylaminobenzaldehyde (DMAB), an alcohol and an acid; (ii) wiping the surface or contacting the solid or liquid sample with the detection wipe; (iii) observing whether the detection wipe exhibits a change in color; and (iv) determining whether the target explosive or illicit drug substance is present on the surface or in the solid or liquid sample based on whether the detection wipe exhibits a color change.

A method of determining whether a target explosive or illicit drug substance is present on a surface or in a solid or liquid sample, comprising: (i) providing a detection wipe fabricated from a fibrous substrate; (ii) contacting the detection wipe with the surface or with the solid or liquid sample; (iii) after said contacting, introducing a chemical detection solution onto the wipe, the chemical detection solution selected from the group consisting of: (a) a combination of dimethyl sulfoxide, an alcohol, an acid and diphenylamine; (b) a combination of dimethyl sulfoxide, isopropyl alcohol, sulfuric acid, diphenylamine, dipropylene glycol dimethyl ether, ferric chloride and hydrochloric acid; (c) a combination of ethylene diamine dihydrochloride, dimethyl sulfoxide, tetrabutylammonium hydroxide, isopropyl alcohol, sulfanilamide, butanol, zinc powder and phosphoric acid; (d) a combination of eosin Y reagent dissolved in phosphate buffered and yellow food coloring; (e) a combination of acetaldehyde, sodium nitroprusside and sodium carbonate; (f) a combination of cobalt thiocyanate, glycerin, an acid and optionally chloroform; and (g) a combination of p-dimethylaminobenzaldehyde (DMAB), an alcohol and an acid; (iv) observing whether the detection wipe exhibits a change in color; and (v) determining whether the target explosive or illicit drug substance is present on the surface or in the solid or liquid sample based on whether the detection wipe exhibits a color change.

A method of determining whether a target explosive or illicit drug substance is present on a surface, or in a solid or liquid sample comprising: (i) dispensing a detection wipe comprising a fibrous substrate from a container that contains multiple detection wipes and that is operable to dispense wipes individually; (ii) introducing a chemical detection solution onto the wipe to provide an impregnated wipe, the chemical detection solution selected from the group consisting of: (a) a combination of dimethyl sulfoxide, an alcohol, an acid and diphenylamine; (b) a combination of dimethyl sulfoxide, isopropyl alcohol, sulfuric acid, diphenylamine, dipropylene glycol dimethyl ether, ferric chloride and hydrochloric acid; (c) a combination of ethylene diamine dihydrochloride, dimethyl sulfoxide, tetrabutylammonium hydroxide, isopropyl alcohol, sulfanilamide, butanol, zinc powder and phosphoric acid; (d) a combination of eosin Y reagent dissolved in phosphate buffered and yellow food coloring; (e) a combination of acetaldehyde, sodium nitroprusside and sodium carbonate; (f) a combination of cobalt thiocyanate, glycerin, an acid and optionally chloroform; and (g) a combination of p-dimethylaminobenzaldehyde (DMAB), an alcohol and an acid; (iii) wiping the surface or contacting the solid or liquid sample with the impregnated detection wipe; (iv) observing whether the impregnated detection wipe exhibits a change in color; and (v) determining whether the target explosive or illicit drug substance is present on the surface or in the solid or liquid sample based on whether the impregnated detection wipe exhibits a color change.

A method of determining whether a target explosive or illicit drug substance is present on a surface or in a solid or liquid sample, comprising: (i) dispensing a detection wipe comprising a fibrous substrate from a container that contains multiple detection wipes and that is operable to dispense wipes individually; (ii) wiping the surface or contacting the solid or liquid sample with the detection wipe; (iii) after said wiping or contacting, introducing a chemical detection solution onto the wipe, the chemical detection solution selected from the group consisting of: (a) a combination of dimethyl sulfoxide, an alcohol, an acid and diphenylamine; (b) a combination of dimethyl sulfoxide, isopropyl alcohol, sulfuric acid, diphenylamine, dipropylene glycol dimethyl ether, ferric chloride and hydrochloric acid; (c) a combination of ethylene diamine dihydrochloride, dimethyl sulfoxide, tetrabutylammonium hydroxide, isopropyl alcohol, sulfanilamide, butanol, zinc powder and phosphoric acid; (d) a combination of eosin Y reagent dissolved in phosphate buffered and yellow food coloring; (e) a combination of acetaldehyde, sodium nitroprusside and sodium carbonate; (f) a combination of cobalt thiocyanate, glycerin, an acid and optionally chloroform; and (g) a combination of p-dimethylaminobenzaldehyde (DMAB), an alcohol and an acid; (iv) observing whether the detection wipe exhibits a change in color; and (v) determining whether the target explosive or illicit drug substance is present on the surface or in the solid or liquid sample based on whether the detection wipe exhibits a color change.

A method in accordance with any other embodiment described herein wherein the chemical detection solution includes dimethyl sulfoxide, isopropyl alcohol, sulfuric acid and diphenylamine and where the target substance is chlorate.

A method in accordance with any other embodiment described herein wherein the chemical detection solution has a pH of from about 1.0 to about 6.0 or from about 2.5 to about 5.0.

A method in accordance with any other embodiment described herein wherein the chemical detection solution comprises from about 6 to about 9% dimethyl sulfoxide (by weight), from about 4 to about 8% isopropyl alcohol (by weight), from about 0.1 to about 2% diphenylamine (by weight) and from about 80% to about 90.0% by weight of sulfuric acid.

A method in accordance with any other embodiment described herein wherein the sulfuric acid has a concentration of from about 1.5M to about 11.0M or from about 5.4M to about 5.8M.

A method in accordance with any other embodiment described herein wherein the sulfuric acid has a concentration of about 2.55M or about 5.6M.

A method in accordance with any other embodiment described herein wherein the chemical detection solution comprises about 7.4% dimethyl sulfoxide (by weight), about 6.0% isopropyl alcohol (by weight), about 0.5% diphenylamine (by weight) and about 86.0% by weight of sulfuric acid.

A method in accordance with any other embodiment described herein wherein the chemical detection solution includes dimethyl sulfoxide, isopropyl alcohol, sulfuric acid, diphenylamine, dipropylene glycol dimethyl ether, ferric chloride and hydrochloric acid, and where the target substance is peroxide.

A method in accordance with any other embodiment described herein wherein the chemical detection solution includes ethylene diamine dihydrochloride, dimethyl sulfoxide, tetrabutylammonium hydroxide, isopropyl alcohol, sulfanilamide, butanol, zinc powder and phosphoric acid, and where the target substance is a nitro explosive substance.

A method in accordance with any other embodiment described herein wherein the chemical detection solution includes eosin Y reagent dissolved in phosphate buffered and yellow food coloring, and where the target substance is fentanyl.

A method in accordance with any other embodiment described herein wherein the chemical detection solution includes acetaldehyde, sodium nitroprusside and sodium carbonate, and where the target substance is methamphetamine.

A method in accordance with any other embodiment described herein wherein the chemical detection solution includes cobalt thiocyanate, glycerin, an acid and optionally chloroform, and where the target substance is cocaine.

A method in accordance with any other embodiment described herein wherein the chemical detection solution includes p-dimethylaminobenzaldehyde (DMAB), an alcohol and an acid, and where the target substance is a synthetic cannabimimetic drug containing an aminoalkyl indole group.

A method in accordance with any other embodiment described herein, further comprising, after said observing: (i) introducing a second chemical detection solution onto the wipe, the second chemical detection solution including an aqueous solution of methylene blue; (ii) after said introducing the second chemical detection solution onto the wipe, observing whether the second chemical detection solution exhibits a change in color; and (iii) determining whether perchlorate is present on the surface based on whether the second chemical detection solution exhibits a color change to violet after introduction of the second chemical detection solution onto the wipe.

A method in accordance with any other embodiment described herein the second chemical detection solution comprises an aqueous solution of methylene blue having a concentration of from about 0.1% to about 1.0%, a concentration of from about 0.2% to about 0.5% or a concentration of about 0.25%.

A method of detecting the presence of a nitro explosive substance, comprising: (i) providing a packaged detection wipe embodiment for a nitro explosive substance as described herein; (ii) activating the wipe by applying compressive force to one of the first and second compartments to breach the frangible seal, thereby mixing the first and second precursor solutions within the fibrous substrate; (iii) wiping a surface or touching a solid or liquid sample with the detection wipe; (iv) observing whether the detection wipe exhibits a change in color; and (v) determining whether a nitro explosive substance is present on the surface based on whether the detection wipe exhibits a color change.

A method of detecting the presence of a methamphetamine, comprising: (i) providing a packaged detection wipe embodiment for a methamphetamine as described herein; (ii) activating the wipe by applying compressive force to one of the first and second compartments to breach the frangible seal, thereby mixing the first and second precursor solutions within the fibrous substrate; (iii) wiping a surface or touching a solid or liquid sample with the detection wipe; (iv) observing whether the detection wipe exhibits a change in color; and (v) determining whether methamphetamine is present on the surface based on whether the detection wipe exhibits a color change.

A method of detecting the presence of a peroxide, comprising: (i) providing a packaged detection wipe embodiment for a peroxide as described herein; (ii) activating the wipe by applying compressive force to one of the first and second compartments to breach the frangible seal, thereby mixing the first and second precursor solutions within the fibrous substrate; (iii) wiping a surface or touching a solid or liquid sample with the detection wipe; (iv) observing whether the detection wipe exhibits a change in color; and (v) determining whether a peroxide is present on the surface based on whether the detection wipe exhibits a color change.

A method of detecting the presence of a chlorate or a perchlorate, comprising: (i) providing a packaged detection wipe embodiment for a chlorate or a perchlorate as described herein; (ii) activating the wipe by applying compressive force to one of the first and second compartments to breach the frangible seal; (iii) permitting the second chemical detection solution to flow into the first compartment and wet a first portion of the fibrous substrate but not a second portion of the fibrous substrate; (iv) wiping a surface or touching a solid or liquid sample with the detection wipe; (v) observing whether the first portion of the detection wipe exhibits a change in color and whether the second portion of the detection wipe exhibits a change in color; (vi) determining whether a chlorate is present on the surface based on whether the first portion of the detection wipe exhibits a color change; and (vii) determining whether a perchlorate is present on the surface based on whether the second portion of the detection wipe exhibits a color change.

A method of determining whether an explosive substance or an illicit drug substance is present on a surface or in a solid or liquid sample, comprising: (i) providing a first packaged wipe embodiment as described herein that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group; (ii) wiping the surface with the first detection wipe; (iii) observing whether the first detection wipe exhibits a change in color; (iv) determining whether the first target substance is present on the surface based on whether the first detection wipe exhibits a color change; (v) providing a second detection wipe embodiment as described herein that is operable to undergo a chemical reaction or a series of chemical reactions to produce a compound having a characteristic visible color when the solution contacts a second target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the second target substance is different than the first target substance; (vi) wiping the surface with the second detection wipe; (vii) observing whether the second detection wipe exhibits a change in color; and (viii) determining whether the second target substance is present on the surface based on whether the second detection wipe exhibits a color change.

A method in accordance with any other embodiment described herein, further comprising: (i) providing a third detection wipe embodiment as described herein that is operable to undergo a chemical reaction or a series of chemical reactions to produce a compound having a characteristic visible color when the solution contacts a third target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the third target substance is different than the first and second target substances; (ii) wiping the surface with the third detection wipe; (iii) observing whether the third detection wipe exhibits a change in color; and (iv) determining whether the third target substance is present on the surface based on whether the third detection wipe exhibits a color change.

A kit to test for a plurality of diverse target substances on a surface, the target substances selected from explosive substances and illicit drug substances, the kit comprising at least two packaged wipes selected from the group consisting of: (i) a first packaged wipe embodiment as described herein that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group; (ii) a second packaged wipe embodiment as described herein that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the second target substance is different than the first target substance; and (iii) a third packaged wipe embodiment as described herein that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the third target substance is different than the first and second target substances.

A kit to test for a plurality of diverse explosive substances on a surface, the kit comprising: (i) a wipe contained within a chamber of a sealed package, the wipe impregnated with a first chemical detection solution comprising dimethyl sulfoxide, an alcohol, an acid and diphenylamine; wherein the first chemical detection solution are operable to produce compounds having a visible color when the solution contacts a chlorate; (ii) a container; and (iii) a mixture contained within the container, the mixture comprising dipropylene glycol dimethyl ether, ferric chloride, and hydrochloric acid; wherein admixture of the first chemical detection solution with the mixture produces a second chemical detection solution operable to produce a compound having a visible color when the solution contacts hydrogen peroxide or an organic peroxide such as TATP or MEKP.

While exemplary embodiments incorporating the principles of the present application have been disclosed herein, the present application is not limited to the disclosed embodiments. Instead, this application is intended to cover any variations, uses, or adaptations of the application using its general principles. Further, this application is intended to cover such departures from the present disclosure as come within known or customary practice in the art to which this present application pertains and which fall within the limits of the appended claims.

The terminology used herein is for the purpose of describing illustrative embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method actions, processes, and operations described herein are not to be construed as necessarily requiring their performance in the particular order discussed or illustrated, unless specifically identified as an order of performance. It is also to be understood that additional or alternative actions or operations may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer, or section from another region, layer, or section. Terms such as “first,” “second,” and other numerical terms when used herein do not imply a sequence or order unless clearly indicated by the context. Thus, a first element, component, region, layer, or section discussed herein could be termed a second element, component, region, layer, or section without departing from the teachings of the example embodiments.

What is claimed is:

1. A packaged chemical detection wipe operable to detect the presence of an explosive substance or an illicit drug substance, comprising:
a flexible package including a first seal surrounding a first compartment and a second compartment and including a second seal between the first compartment and the second compartment,
a fibrous substrate positioned in the first compartment;
a first precursor component of a chemical detection solution positioned in the first compartment and impregnated within the fibrous substrate; and
a second precursor component of the chemical detection solution positioned in the second compartment;
wherein the second seal is a frangible seal configured to be breached upon application to the first compartment or the second compartment a compressive force of a magnitude sufficient to breach the frangible seal but insufficient to breach the first seal, thereby permitting the first precursor component and the second precursor component to mix;
wherein mixture of the first precursor component and the second precursor component produces a chemical detection solution operable, when contacted with the explosive substance or the illicit drug substance, to undergo a chemical reaction or a series of chemical reactions to produce a compound having a characteristic visible color.

2. The packaged chemical detection wipe of claim 1 wherein the chemical detection wipe comprises a nitro-detection wipe; wherein the first precursor component comprises dimethyl sulfoxide, tetrabutylammonium hydroxide, ethylenediamine dihydrochloride and isopropyl alcohol; and wherein the second precursor component comprises sulfanilamide and phosphoric acid.

3. The packaged chemical detection wipe of claim 1 wherein the chemical detection wipe comprises a methamphetamine-detection wipe; wherein the first precursor component comprises an aqueous solution of sodium nitroprusside and acetaldehyde; and wherein the second precursor component comprises an aqueous solution of sodium carbonate.

4. The packaged chemical detection wipe of claim 1 wherein the chemical detection wipe comprises a peroxide-detection wipe; wherein the first precursor component comprises dipropylene glycol dimethyl ether, ferric chloride, and hydrochloric acid; and wherein the second precursor component comprises dimethyl sulfoxide, isopropyl alcohol, diphenylamine, and sulfuric acid.

5. The packaged chemical detection wipe of claim 4 wherein the second precursor component has a pH of from about 1.0 to about 6.0 or from about 2.5 to about 5.0.

6. The packaged chemical detection wipe of claim 4 wherein the second precursor component comprises from about 6 to about 9% dimethyl sulfoxide (by weight), from about 4 to about 8% isopropyl alcohol (by weight), from about 0.1 to about 2% diphenylamine (by weight) and from about 80% to about 90.0% by weight of sulfuric acid.

7. The packaged chemical detection wipe of claim 4 wherein the second precursor component comprises about 7.4% dimethyl sulfoxide (by weight), about 6.0% isopropyl alcohol (by weight), about 0.5% diphenylamine (by weight) and about 86.0% by weight of sulfuric acid.

8. The packaged chemical detection wipe of claim 6 or claim 7 wherein the sulfuric acid has a concentration of from about 1.5M to about 11.0M or from about 5.4M to about 5.8M.

9. The packaged chemical detection wipe of claim 6 or claim 7 wherein the sulfuric acid has a concentration of about 2.55M or about 5.6M.

10. A packaged chemical detection wipe operable to detect the presence of a chlorate explosive substance and a perchlorate explosive substance, comprising:
a flexible package including a first seal surrounding a first compartment and a second compartment and including a second seal between the first compartment and the second compartment;
a fibrous substrate positioned in the first compartment;
a first chemical detection solution positioned in the first compartment and impregnated within the fibrous substrate; and
a second chemical detection solution positioned in the second compartment;
wherein the second seal is a frangible seal configured to be breached upon application to the first compartment or the second compartment a compressive force of a magnitude sufficient to breach the frangible seal but insufficient to breach the first seal, thereby permitting the second chemical detection solution to wet a portion of the wipe;
wherein the first chemical detection solution is operable, when contacted with a chlorate compound, to undergo a chemical reaction or a series of chemical reactions to produce a compound having a characteristic visible color; and
wherein the second chemical detection solution is operable, when contacted with a perchlorate compound, to undergo a chemical reaction or a series of chemical reactions to produce a compound having a characteristic visible color.

11. The packaged chemical detection wipe of claim 10 wherein the first chemical detection solution comprises dimethyl sulfoxide, isopropyl alcohol, sulfuric acid and diphenylamine; and wherein the second chemical detection solution comprises an aqueous solution of methylene blue.

12. The packaged chemical detection wipe of claim 10 wherein the second chemical detection solution comprises an aqueous solution of methylene blue having a concentration of from about 0.1% to about 1.0%, a concentration of from about 0.2% to about 0.5% or a concentration of about 0.25%.

13. A packaged chemical detection wipe operable to detect the presence of fentanyl, comprising:
a flexible package including a seal surrounding a compartment;
a fibrous substrate positioned in the compartment; and
a fluid chemical detection solution positioned in the compartment and impregnated into the substrate, the chemical detection solution operable to undergo a chemical reaction or a series of chemical reactions to produce a compound having a characteristic visible color when the solution contacts fentanyl;
wherein the chemical detection solution comprises an aqueous buffered solution of eosin Y and optionally further comprises yellow food coloring.

14. A packaged chemical detection wipe operable to detect the presence of cocaine, comprising:
a flexible package including a seal surrounding a compartment;
a fibrous substrate positioned in the compartment; and
a fluid chemical detection solution positioned in the compartment and impregnated into the substrate, the chemical detection solution operable to undergo a chemical reaction or a series of chemical reactions to produce a compound having a characteristic visible color when the solution contacts cocaine;
wherein the chemical detection solution comprises cobalt thiocyanate, 10% acetic acid, glycerin and optionally one or both of hydrochloric acid and chloroform.

15. A packaged chemical detection wipe operable to detect the presence of an illicit substance, comprising:
a flexible package including a seal surrounding a compartment;
a fibrous substrate positioned in the compartment; and
a fluid chemical detection solution positioned in the compartment and impregnated into the substrate, the chemical detection solution operable to undergo a chemical reaction or a series of chemical reactions to produce a compound having a characteristic visible color when the solution contacts the illicit substance;
wherein the chemical detection solution comprises Ehrlich reagent.

16. The packaged chemical detection wipe in accordance with claim 15 wherein the Ehrlich reagent comprises p-dimethylaminobenzaldehyde (DMAB) dissolved in a mixture of an alcohol and an acid.

17. The packaged chemical detection wipe in accordance with claim 15 wherein the Ehrlich reagent is prepared by dissolving from about 0.5 to about 2.0 grams of DMAB in a solution of about 50 mL of 95% ethanol and about 50 mL of concentrated hydrochloric acid.

18. The packaged chemical detection wipe in accordance with claim 15 wherein the illicit drug comprises a psychoactive substances such as K2, spice or LSD.

19. The packaged chemical detection wipe of any of claims 1-18 wherein the fibrous substrate comprises a plurality of fibers selected from the group consisting of cellulose, polyamides, polyesters, polyethylenes, polypropylenes, polyacrylics, cellulose acetate, polylactic acid, silk, wool, glass, polyaramids, and combinations thereof.

20. The packaged chemical detection wipe of any of claims 1-18 wherein the fibrous substrate is a nonwoven material having multiple layers formed from fibers selected from the group consisting of polyolefin, polypropylene, polyethylene, ethylene copolymers and propylene copolymers.

21. The packaged chemical detection wipe of any of claims 1-18 wherein the fibrous substrate is an absorbent nonwoven material formed from a blend of spunbonded fibers.

22. The packaged chemical detection wipe of claim 21 wherein the blend of spunbonded fibers is selected from the group consisting of polypropylene, polyester and wood pulp.

23. A method of fabricating a detection system for cocaine, the method comprising:
providing a chemical detection solution that includes cobalt thiocyanate, 10% acetic acid, glycerin, and optionally one or both of hydrochloric acid and chloroform;
providing a fibrous substrate; and
impregnating the fibrous substrate with the chemical detection solution to provide an impregnated substrate.

24. The method of claim 23 wherein the impregnated substrate is configured to be wiped over a surface or touched to a solid or liquid sample for detection of the presence or absence of an illicit substance on the surface or in the sample.

25. The method of claim 23, further comprising placing the impregnated substrate in a sealed package.

26. The method of claim 23 wherein the impregnated substrate is configured to be removed from the sealed package and wiped over a surface or touched to a solid or liquid sample for detection of the presence or absence of cocaine on the surface or in the sample.

27. A method of fabricating chemical detection wipe operable to detect an explosive substance or an illicit drug, the method comprising:
providing a chemical detection solution selected from the group consisting of:
a combination of dimethyl sulfoxide, an alcohol, an acid and diphenylamine;
a combination of dimethyl sulfoxide, isopropyl alcohol, sulfuric acid, diphenylamine, dipropylene glycol dimethyl ether, ferric chloride and hydrochloric acid;
a combination of ethylene diamine dihydrochloride, dimethyl sulfoxide, tetrabutylammonium hydroxide, isopropyl alcohol, sulfanilamide, butanol, zinc powder and phosphoric acid;
a combination of eosin Y reagent dissolved in phosphate buffered and yellow food coloring;
a combination of acetaldehyde, sodium nitroprusside and sodium carbonate;
a combination of cobalt thiocyanate, glycerin, an acid and optionally chloroform; and
a combination of p-dimethylaminobenzaldehyde (DMAB), an alcohol and an acid;
providing a fibrous substrate; and
impregnating the fibrous substrate with the chemical detection solution to provide an impregnated substrate.

28. The method of claim 27 wherein the impregnated substrate is configured to be wiped over a surface or contacted with a solid or liquid sample for detection of the presence or absence of an explosive substance or an illicit drug on the surface or in the solid or liquid sample.

29. The method of claim 27 wherein the plurality of compounds and the substrate are contained within a sealed package.

30. The method of claim 27, further comprising placing the impregnated substrate in a sealed package.

31. The method of claim 30 wherein the impregnated substrate is configured to be removed from the sealed package and wiped over a surface or contacted with a solid or liquid sample for detection of the presence or absence of an explosive substance or an illicit drug on the surface or in the solid or liquid sample.

32. A method of determining whether a target explosive or illicit drug substance is present on a surface or in a solid or liquid sample, comprising:
providing a detection wipe fabricated from a fibrous substrate impregnated with a chemical detection solution, the chemical detection solution selected from the group consisting of:
a combination of dimethyl sulfoxide, an alcohol, an acid and diphenylamine;
a combination of dimethyl sulfoxide, isopropyl alcohol, sulfuric acid, diphenylamine, dipropylene glycol dimethyl ether, ferric chloride and hydrochloric acid;
a combination of ethylene diamine dihydrochloride, dimethyl sulfoxide, tetrabutylammonium hydroxide, isopropyl alcohol, sulfanilamide, butanol, zinc powder and phosphoric acid;
a combination of eosin Y reagent dissolved in phosphate buffered and yellow food coloring;
a combination of acetaldehyde, sodium nitroprusside and sodium carbonate;
a combination of cobalt thiocyanate, glycerin, an acid and optionally chloroform; and
a combination of p-dimethylaminobenzaldehyde (DMAB), an alcohol and an acid;
wiping the surface or contacting the solid or liquid sample with the detection wipe;
observing whether the detection wipe exhibits a change in color; and
determining whether the target explosive or illicit drug substance is present on the surface or in the solid or liquid sample based on whether the detection wipe exhibits a color change.

33. A method of determining whether a target explosive or illicit drug substance is present on a surface or in a solid or liquid sample, comprising:
providing a detection wipe fabricated from a fibrous substrate;
contacting the detection wipe with the surface or with the solid or liquid sample;
after said contacting, introducing a chemical detection solution onto the wipe, the chemical detection solution selected from the group consisting of:
a combination of dimethyl sulfoxide, an alcohol, an acid and diphenylamine;
a combination of dimethyl sulfoxide, isopropyl alcohol, sulfuric acid, diphenylamine, dipropylene glycol dimethyl ether, ferric chloride and hydrochloric acid;
a combination of ethylene diamine dihydrochloride, dimethyl sulfoxide, tetrabutylammonium hydroxide, isopropyl alcohol, sulfanilamide, butanol, zinc powder and phosphoric acid;
a combination of eosin Y reagent dissolved in phosphate buffered and yellow food coloring;
a combination of acetaldehyde, sodium nitroprusside and sodium carbonate;
a combination of cobalt thiocyanate, glycerin, an acid and optionally chloroform; and
a combination of p-dimethylaminobenzaldehyde (DMAB), an alcohol and an acid;
observing whether the detection wipe exhibits a change in color; and
determining whether the target explosive or illicit drug substance is present on the surface or in the solid or liquid sample based on whether the detection wipe exhibits a color change.

34. A method of determining whether a target explosive or illicit drug substance is present on a surface, or in a solid or liquid sample comprising:
dispensing a detection wipe comprising a fibrous substrate from a container that contains multiple detection wipes and that is operable to dispense wipes individually;
introducing a chemical detection solution onto the wipe to provide an impregnated wipe, the chemical detection solution selected from the group consisting of:
a combination of dimethyl sulfoxide, an alcohol, an acid and diphenylamine;
a combination of dimethyl sulfoxide, isopropyl alcohol, sulfuric acid, diphenylamine, dipropylene glycol dimethyl ether, ferric chloride and hydrochloric acid;
a combination of ethylene diamine dihydrochloride, dimethyl sulfoxide, tetrabutylammonium hydroxide, isopropyl alcohol, sulfanilamide, butanol, zinc powder and phosphoric acid;
a combination of eosin Y reagent dissolved in phosphate buffered and yellow food coloring;
a combination of acetaldehyde, sodium nitroprusside and sodium carbonate;
a combination of cobalt thiocyanate, glycerin, an acid and optionally chloroform; and
a combination of p-dimethylaminobenzaldehyde (DMAB), an alcohol and an acid;
wiping the surface or contacting the solid or liquid sample with the impregnated detection wipe;
observing whether the impregnated detection wipe exhibits a change in color; and
determining whether the target explosive or illicit drug substance is present on the surface or in the solid or liquid sample based on whether the impregnated detection wipe exhibits a color change.

35. A method of determining whether a target explosive or illicit drug substance is present on a surface or in a solid or liquid sample, comprising:
dispensing a detection wipe comprising a fibrous substrate from a container that contains multiple detection wipes and that is operable to dispense wipes individually;
wiping the surface or contacting the solid or liquid sample with the detection wipe;
after said wiping or contacting, introducing a chemical detection solution onto the wipe, the chemical detection solution selected from the group consisting of:
a combination of dimethyl sulfoxide, an alcohol, an acid, and diphenylamine;
a combination of dimethyl sulfoxide, isopropyl alcohol, sulfuric acid, diphenylamine, dipropylene glycol dimethyl ether, ferric chloride and hydrochloric acid;
a combination of ethylene diamine dihydrochloride, dimethyl sulfoxide, tetrabutylammonium hydroxide, isopropyl alcohol, sulfanilamide, butanol, zinc powder and phosphoric acid;
a combination of eosin Y reagent dissolved in phosphate buffered and yellow food coloring;
a combination of acetaldehyde, sodium nitroprusside and sodium carbonate;
a combination of cobalt thiocyanate, glycerin, an acid and optionally chloroform; and
a combination of p-dimethylaminobenzaldehyde (DMAB), an alcohol and an acid;
observing whether the detection wipe exhibits a change in color; and
determining whether the target explosive or illicit drug substance is present on the surface or in the solid or liquid sample based on whether the detection wipe exhibits a color change.

36. The method of any of claims 32, 33, 34 and 35 wherein the chemical detection solution includes dimethyl sulfoxide, isopropyl alcohol, sulfuric acid and diphenylamine and where the target substance is chlorate.

37. The method of claim 36 wherein the chemical detection solution has a pH of from about 1.0 to about 6.0 or from about 2.5 to about 5.0.

38. The method of claim 36 wherein the chemical detection solution comprises from about 6 to about 9% dimethyl sulfoxide (by weight), from about 4 to about 8% isopropyl alcohol (by weight), from about 0.1 to about 2% diphenylamine (by weight) and from about 80% to about 90.0% by weight of sulfuric acid.

39. The method of claim 38 wherein the sulfuric acid has a concentration of from about 1.5M to about 11.0M or from about 5.4M to about 5.8M.

40. The method of claim 38 wherein the sulfuric acid has a concentration of about 2.55M or about 5.6M.

41. The method of claim 36 wherein the chemical detection solution comprises about 7.4% dimethyl sulfoxide (by weight), about 6.0% isopropyl alcohol (by weight), about 0.5% diphenylamine (by weight) and about 86.0% by weight of sulfuric acid.

42. The method of claim 41 wherein the sulfuric acid has a concentration of from about 1.5M to about 11.0M or from about 5.4M to about 5.8M.

43. The method of claim 41 wherein the sulfuric acid has a concentration of about 2.55M or about 5.6M.

44. The method of any of claims 27, 32, 33, 34 and 35 wherein the chemical detection solution includes dimethyl sulfoxide, isopropyl alcohol, sulfuric acid, diphenylamine, dipropylene glycol dimethyl ether, ferric chloride and hydrochloric acid, and where the target substance is peroxide.

45. The method of any of claims 27, 32, 33, 34 and 35 wherein the chemical detection solution includes ethylene diamine dihydrochloride, dimethyl sulfoxide, tetrabutylammonium hydroxide, isopropyl alcohol, sulfanilamide, butanol, zinc powder and phosphoric acid, and where the target substance is a nitro explosive substance.

46. The method of any of claims 27, 32, 33, 34 and 35 wherein the chemical detection solution includes eosin Y reagent dissolved in phosphate buffered and yellow food coloring, and where the target substance is fentanyl.

47. The method of any of claims 27, 32, 33, 34 and 35 wherein the chemical detection solution includes acetaldehyde, sodium nitroprusside and sodium carbonate, and where the target substance is methamphetamine.

48. The method of any of claims 27, 32, 33, 34 and 35 wherein the chemical detection solution includes cobalt thiocyanate, glycerin, an acid and optionally chloroform, and where the target substance is cocaine.

49. The method of any of claims 27, 32, 33, 34 and 35 wherein the chemical detection solution includes p-dimethylaminobenzaldehyde (DMAB), an alcohol and an acid, and where the target substance is a synthetic cannabimimetic drug containing an aminoalkyl indole group.

50. The method of any of claim 36, further comprising, after said observing:
introducing a second chemical detection solution onto the wipe, the second chemical detection solution including an aqueous solution of methylene blue;
after said introducing the second chemical detection solution onto the wipe, observing whether the second chemical detection solution exhibits a change in color; and
determining whether perchlorate is present on the surface based on whether the second chemical detection solution exhibits a color change to violet after introduction of the second chemical detection solution onto the wipe.

51. The method of claim 50 wherein the second chemical detection solution comprises an aqueous solution of methylene blue having a concentration of from about 0.1% to about 1.0%, a concentration of from about 0.2% to about 0.5% or a concentration of about 0.25%.

52. A method of detecting the presence of a nitro explosive substance, comprising:
providing a packaged detection wipe in accordance with claim 2;
activating the wipe by applying compressive force to one of the first and second compartments to breach the frangible seal, thereby mixing the first and second precursor solutions within the fibrous substrate;
wiping a surface or touching a solid or liquid sample with the detection wipe;
observing whether the detection wipe exhibits a change in color; and
determining whether a nitro explosive substance is present on the surface based on whether the detection wipe exhibits a color change.

53. A method of detecting the presence of a methamphetamine, comprising:
providing a packaged detection wipe in accordance with claim 3;
activating the wipe by applying compressive force to one of the first and second compartments to breach the frangible seal, thereby mixing the first and second precursor solutions within the fibrous substrate;
wiping a surface or touching a solid or liquid sample with the detection wipe;
observing whether the detection wipe exhibits a change in color; and
determining whether methamphetamine is present on the surface based on whether the detection wipe exhibits a color change.

54. A method of detecting the presence of a peroxide, comprising:
providing a packaged detection wipe in accordance with claim 4;
activating the wipe by applying compressive force to one of the first and second compartments to breach the frangible seal, thereby mixing the first and second precursor solutions within the fibrous substrate;
wiping a surface or touching a solid or liquid sample with the detection wipe;
observing whether the detection wipe exhibits a change in color; and
determining whether a peroxide is present on the surface based on whether the detection wipe exhibits a color change.

55. A method of detecting the presence of a chlorate or a perchlorate, comprising:
providing a packaged detection wipe in accordance with claim 10;
activating the wipe by applying compressive force to one of the first and second compartments to breach the frangible seal;
permitting the second chemical detection solution to flow into the first compartment and wet a first portion of the fibrous substrate but not a second portion of the fibrous substrate;
wiping a surface or touching a solid or liquid sample with the detection wipe;
observing whether the first portion of the detection wipe exhibits a change in color and whether the second portion of the detection wipe exhibits a change in color;
determining whether a chlorate is present on the surface based on whether the first portion of the detection wipe exhibits a color change; and
determining whether a perchlorate is present on the surface based on whether the second portion of the detection wipe exhibits a color change.

56. A method of determining whether an explosive substance or an illicit drug substance is present on a surface or in a solid or liquid sample, comprising:
providing a first packaged wipe in accordance with any of claims 1-16 that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group;
wiping the surface with the first detection wipe;
observing whether the first detection wipe exhibits a change in color;
determining whether the first target substance is present on the surface based on whether the first detection wipe exhibits a color change;
providing a second detection wipe in accordance with any of claims 1-16 that is operable to undergo a chemical reaction or a series of chemical reactions to produce a compound having a characteristic visible color when the solution contacts a second target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the second target substance is different than the first target substance;
wiping the surface with the second detection wipe;
observing whether the second detection wipe exhibits a change in color; and
determining whether the second target substance is present on the surface based on whether the second detection wipe exhibits a color change.

57. The method of claim 56, further comprising:
providing a third detection wipe in accordance with any of claims 1-16 that is operable to undergo a chemical reaction or a series of chemical reactions to produce a compound having a characteristic visible color when the solution contacts a third target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the third target substance is different than the first and second target substances;
wiping the surface with the third detection wipe;
observing whether the third detection wipe exhibits a change in color; and
determining whether the third target substance is present on the surface based on whether the third detection wipe exhibits a color change.

58. A kit to test for a plurality of diverse target substances on a surface, the target substances selected from explosive substances and illicit drug substances, the kit comprising at least two packaged wipes selected from the group consisting of:
a first packaged wipe in accordance with any of claims 1-16 that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group;
a second packaged wipe in accordance with any of claims 1-16 that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the second target substance is different than the first target substance; and
a third packaged wipe in accordance with any of claims 1-16 that is operable to undergo a chemical reaction or a series of chemical reactions to produce a compound having a characteristic visible color when the solution contacts a first target substance selected from the group consisting of a nitro- or nitrate-containing explosive substance, a methamphetamine, a peroxide, a chlorate, a perchlorate, a fentanyl, a cocaine and a synthetic cannabimimetic containing an aminoalkyl indole group, wherein the third target substance is different than the first and second target substances.

59. A kit to test for a plurality of diverse explosive substances on a surface, the kit comprising:
a wipe contained within a chamber of a sealed package, the wipe impregnated with a first chemical detection solution comprising dimethyl sulfoxide, an alcohol, an acid and diphenylamine; wherein the first chemical detection solution are operable to produce compounds having a visible color when the solution contacts a chlorate;
a container; and
a mixture contained within the container, the mixture comprising dipropylene glycol dimethyl ether, ferric chloride, and hydrochloric acid; wherein admixture of the first chemical detection solution with the mixture produces a second chemical detection solution operable to produce a compound having a visible color when the solution contacts hydrogen peroxide or an organic peroxide such as TATP or MEKP.
